(19) United States
(12) Patent Application Publication
(10) Pub. No.: US 2025/0363631 A1
(43) Pub. Date: Nov. 27, 2025

(54) DEEP LEARNING-BASED DIAGNOSTIC QUALITY PREDICTION DURING MAGNETIC RESONANCE ELASTOGRAPHY DATA ACQUISITION

(71) Applicants: Georgia Tech Research Corporation, Atlanta, GA (US); Icahn School of Medicine at Mount Sinai, New York, NY (US)

(72) Inventors: Jun Ueda, Atlanta, GA (US); Heriberto Andres Nieves-Vazquez, Atlanta, GA (US)

(21) Appl. No.: 19/215,976

(22) Filed: May 22, 2025

Related U.S. Application Data
(60) Provisional application No. 63/650,665, filed on May 22, 2024.

(51) Int. Cl. G06T 7/00; G06T 7/11; G06T 11/00; G06V 10/764; G16H 30/20; G16H 50/20

(52) U.S. Cl. CPC G06T 7/0012; G06T 7/11; G06T 11/005; G06V 10/765; G16H 30/20; G16H 50/20; G06T 2207/10088

(57) ABSTRACT

Disclosed are systems and method for automated magnetic resonance elastography (MRE) quality control and stiffness measurements. The exemplary systems and methods described herein utilize deep learning (DL) to reduce inter-observer variability, improve processing time and workflow constraints, and assist operators with troubleshooting based on artifact sources.

### RELATED APPLICATION

This U.S. application claims priority to, and the benefit of, U.S. Provisional Patent Application No. 63/650,665, filed May 22, 2024, entitled “DEEP LEARNING-BASED DIAGNOSTIC QUALITY PREDICTION DURING MAGNETIC RESONANCE ELASTOGRAPHY DATA ACQUISITION,” which is incorporated by reference herein in its entirety.

### GOVERNMENT LICENSE RIGHTS

This invention was made with government support under DK113272 awarded by the National Institutes of Health, and 2039655 awarded by the National Science Foundation. The government has certain rights in the invention.

### BACKGROUND

Magnetic resonance elastography (MRE) is an imaging technique that combines magnetic resonance imaging (MRI) with low-frequency vibrations to quantitatively measure mechanical properties of tissues. From this data, clinicians are able to create a visual map (elastogram) showing body tissue stiffness, which can provide diagnostically relevant information about a patient.

Despite MRE demonstrating excellent precision and test-retest repeatability on stiffness phantoms, images may still result in poor diagnostic/non-diagnostic quality. This can be due to physiological factors (e.g., iron deposition, overweight), mechanical issues (e.g., MRE driver location), patient non-compliance with breath-hold instructions or movement during image acquisition. These factors can lower image confidence for diagnosis, necessitating reacquisition

Thus, there is a benefit to improving imaging and/or measurements using magnetic resonance elastography.

### SUMMARY

Exemplary systems and method are disclosed for automated magnetic resonance elastography (MRE) quality control and stiffness measurements. The exemplary systems and methods disclosed herein utilize deep learning (DL) to reduce inter-observer variability, improve processing time and workflow constraints, and assist operators with troubleshooting based on artifact sources. Thus, the exemplary methods and systems facilitate a significant improvement in the diagnostic efficacy of MRE.

In various aspects, described herein in a method. The method can include, for example, obtaining magnetic resonance elastography (MRE) imaging data of a subject obtained during a MRE procedure; determining, via a trained AI classification model, a quality assessment metric of the MRE imaging data (e.g., one or more image sets of MRE acquired elastograms and associated outputs), wherein the quality assessment metric corresponds to an indication of a diagnostic quality of the MRE images; and screening the MRE imaging data based on the quality assessment metric to provide a filtered data set of MRE imaging data classified as diagnostic quality.

In some aspects, the AI classification model comprises a binary classification model.

In some aspects, the AI classification model comprises at least one of ResNet18, ResNet34, ResNet50, SqueezeNet, MobileNetV2, or a combination thereof.

In some aspects, the AI classification model comprises SqueezeNet.

In some aspects, the AI classification model comprises an explainable AI (XAI) model.

In some aspects, the XAI model is configured to determine, based on features of a non-diagnostic quality image, a predicted artifact source (e.g., magnitude-related artifacts such as presence of an iron deposition, motion artifact, image blurring, and/or poor wave propagation leading to no measurable area despite successful liver delineation).

In some aspects, the method further includes adjusting a collection of a MRE image (e.g., in real-time) based on the predicted artifact source.

In some aspects, the MRE imaging data comprises MRE magnitude images, 2D Fast Fourier transform (FFT) of MRE magnitude images, or a combination thereof.

In some aspects, the method further includes generating, via a trained AI segmentation model, a segmentation mask corresponding to a region of interest within the filtered data set of MRE imaging data.

In some aspects, the segmentation mask is subsequently used to determine a measurable stiffness area within the filtered data set of MRE imaging data.

In some aspects, the measurable stiffness area is determined for the filtered data set of MRE imaging data using an intersection over union (IoU) of the segmentation mask and a thresholded confidence map obtained from the trained AI segmentation model.

In some aspects, the method further includes diagnosing a condition (e.g., liver fibrosis) in the subject based on stiffness values within the measurable stiffness area.

In another aspect, described herein is a system including: a processor; and a memory having instructions stored thereon, wherein execution of the instructions by the processor causes the processor to: obtain magnetic resonance elastography (MRE) imaging data of a subject collected during a MRE procedure; determine, via a trained AI classification model, a quality assessment metric of the MRE imaging data, wherein the quality assessment metric corresponds to an indication of a diagnostic quality of the MRE imaging data; and screen the MRE imaging data based on the quality assessment metric to provide a filtered data set of MRE imaging data classified as diagnostic quality.

In some aspects, the system includes: a MR scanner configured to collect the MRE imaging data of the subject; and an actuator configured to generate shear waves in a tissue of interest of a subject.

In some aspects, the AI classification model comprises a binary classification model.

In some aspects, the AI classification model comprises an explainable AI (XAI) model configured to determine, based on features of a non-diagnostic quality image, a predicted artifact source (e.g., magnitude-related artifacts such as presence of an iron deposition, motion artifact, image blurring, and/or poor wave propagation leading to no measurable area despite successful liver delineation).

In some aspects, the MRE imaging data comprises MRE magnitude images, 2D Fast Fourier transform (FFT) of MRE magnitude images, or a combination thereof.

In some aspects, the system further includes a trained AI segmentation model configured to generate a segmentation mask corresponding to a region of interest within the filtered data set of MRE imaging data.

In some aspects, the trained AI segmentation model is further configured to determine a measurable stiffness area within the filtered data set of MRE imaging data.

In some aspects, the system is further configured to determine the measurable stiffness area for the filtered data set of MRE imaging data using an intersection over union (IoU) of the segmentation mask and a thresholded confidence map obtained from the trained AI segmentation model.

In another aspects, described herein is a system including: a processor; and a memory having instructions stored thereon, wherein execution of the instructions by the processor causes the processor to: receive magnetic resonance elastography (MRE) data comprising an MRI imaging data of a patient acquired while the patient is subject to a low-frequency vibration (e.g., 60 Hz) to create a visual map (e.g., elastogram) that shows stiffness of body tissues; and determine, via a trained AI model, a quality assessment value associated with a quality metric associated with acquisition of the MRE data; wherein the quality assessment value is employed to reject the MRE data or trigger a notification for a re-acquisition of the MRE data from the patient.

In some aspects, the trained AI model comprises at least one of ResNet18, ResNet34, ResNet50, SqueezeNet, MobileNetV2, or a combination thereof.

In some aspects, the trained AI model was trained using confidence map overlaid elastograms (CMOEs) for liver stiffness measurement having quality score labels (e.g., wherein the CMOEs are in grayscale normalized to a pre-defined range and size).

In some aspects, the MRE imaging data was acquired from 2D spin-echo echo-planar imaging (SE-EPI) sequence.

In some aspects, the MRE imaging data was acquired from 2D gradient-echo (GRE) sequence acquired.

Also described is a non-transitory computer readable medium having instructions stored thereon, wherein execution of the instructions by the processor causes the processor to perform any of the methods described herein or operate any of the systems described herein.

Also described herein is a method of operating any of the systems described herein.

Additional advantages of the invention will be set forth in part in the description which follows, and in part will be clear from the description or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF DRAWINGS

The accompanying drawings, which are incorporated in and constitute a part of this specification, illustrate embodiments and, together with the description, serve to explain the principles of the methods and systems.

FIG. 1 shows a diagram of an example system utilizing deep learning for automated magnetic resonance elastography (MRE) quality control and stiffness measurements in accordance with an illustrative embodiment.

FIGS. 2A-2C each show example methods utilizing deep learning for automated magnetic resonance elastography (MRE) quality control and stiffness measurements in accordance with an illustrative embodiment.

FIG. 3 shows an exemplary workflow of the use of deep learning models in connection with MRE scanners for clinical applications according to an illustrative embodiment.

FIGS. 4A-4D show comparisons between the performance and accuracy of (i) the exemplary system and method and (ii) the current state-of-the-art systems and methods, in a first experiment. FIG. 4A shows a comparison of the workflow between the exemplary deep-learning-based (DL-based) image quality classification and the traditional visual inspection. FIG. 4B shows example curated liver MRE datasets, including a non-diagnostic quality confidence map overlaid elastogram (CMOE) obtained using 2D SE-EPI at 1.5 T and a diagnostic quality CMOE obtained using 2D SE-EPI at 3 T. FIG. 4C shows average training/validation accuracies and losses for each deep learning (DL) model across cross-validation iterations. FIG. 4D shows confusion matrices for each cross-validation iteration, where (N) signifies non-diagnostic image quality and (D) signifies diagnostic image quality, using the test dataset (114 slices).

FIGS. 5A-5K show comparisons between the performance and accuracy of (i) the exemplary system and method and (ii) the reference standard systems and methods, in a second experiment. FIG. 5A shows a binary classification model (e.g., SqueezeNet), in a first portion/step of the exemplary system, configured for automated quality control (QC) assessment. FIG. 5B shows a segmentation model, in a second portion/step of the exemplary system, configured for measurable liver area delineation and image overlaying steps to perform DL-assisted liver stiffness measurement. FIG. 5C shows a polygon tool used by an observer for LSM efficiency evaluation. FIG. 5D shows confusion matrices for DL models using only MRE magnitude images, only 2D FFT images, and a combination of MRE magnitude and 2D FFT images. FIG. 5E shows Dice scores between the segmentation masks delineated by the predicted masks of a DL segmentation model (e.g., 2D U-Net) and the reference standard. FIG. 5F shows the distributions of the liver stiffness measurement (LSM) error (in %), comparing the deep learning (DL)-assisted method (e.g., 2D U-Net segmentation model) for diagnostic-quality slices and the reference standard method. FIG. 5G shows an MRE example slice demonstrating a diagnostic-quality MRE magnitude slice, its associated phase slice, and the confidence map overlaid with the elastogram slice. FIG. 5H shows a scatterplot comparing liver stiffness measurements (LSMs) between the deep learning (DL)-assisted method and the reference standard method. FIGS. 5I-5J show Bland-Altman plots for the DL-assisted method the reference standard method. FIG. 5K shows examples of the segmentation and overlaying procedure that can be used to obtain automated stiffness measurements.

FIG. 6 shows the incorporation of the system architecture into existing MRE platforms according to one embodiment.

### DETAILED DESCRIPTION

Each and every feature described herein, and each and every combination of two or more of such features, is included within the scope of the present invention, provided that the features included in such a combination are not mutually inconsistent.

### Definitions

As used herein, the term “subject” refers to any animal (e.g., a mammal), including, but not limited to, humans, non-human primates, rodents, and the like. Typically, the terms “subject” and “patient” are used interchangeably herein in reference to a human subject.

The systems and methods described herein may be used for characterizing properties of tissue or an organ of the subject. The term “tissue” is used herein in its broadest sense and thus shall be understood to include an aggregate of cells usually of a particular kind together with their intercellular substance that form one of the structural materials of an animal including human beings. In general, there are four basic types of tissue in the body of all animals, including the human body and lower multicellular organisms such as insects, and these include nervous tissue, muscle tissue, epidermal, and connective tissue. These comprise all the organs, structures and other contents. It also should be recognized that term “tissue” as used herein shall not be understood to be limited only to one of the types of tissue but also can include a body part that is composed of more than one type of tissue (e.g., muscle tissue and epidermal). As used herein, the term “organ” is to be understood as a collection of tissue joined in a structural unit to serve a common function. The organ may be a human organ. The organ may be any one of the following, for example: intestines, skeleton, kidneys, gall bladder, liver, muscles, arteries, heart, larynx, pharynx, brain, lymph nodes, lungs, spleen bone marrow, stomach, veins, pancreas, and bladder.

The term “imaging data” includes images and data acquired directly from an imaging apparatus, such as a magnetic resonance imaging (MRI) system. As used herein, the term “image” can refer to a two- or three-dimensional image. Similarly, the term “MRE imaging data” may include images of different types which are acquired from a single MRE acquisition (e.g., MRE magnitude images, phase images, wave images, elastogram).

The term “explainable AI” (XAI) refers to methods and techniques in the application of AI technology such that the results of the solution can be understood by human experts. XAI contrasts with non-explainable AI, where machine learning models (MLM) cannot explain why the AI arrived at a specific decision. Hence, there is a need to justify the information and insights generated by an AI system. XAI models can generate a large amount of metadata which gives the evidence and confidence level to end users that can be validated manually.

As used herein, the term “segmentation model” refers to an unsupervised machine learning model that automatically discovers one or more natural groupings (e.g., “segments”) in data. For example, segmentation models may be used to predict a region corresponding to a target structure (e.g., boundaries of an organ, such as a liver, of a patient) from a medical image.

All patents and publications mentioned in the specification are indicative of the levels of skill of those skilled in the art to which the invention pertains. References cited herein are incorporated by reference herein in their entirety to indicate the state of the art as of their filing date, and it is intended that this information can be employed herein, if needed, to exclude specific embodiments that are in the prior art.

### Example Systems

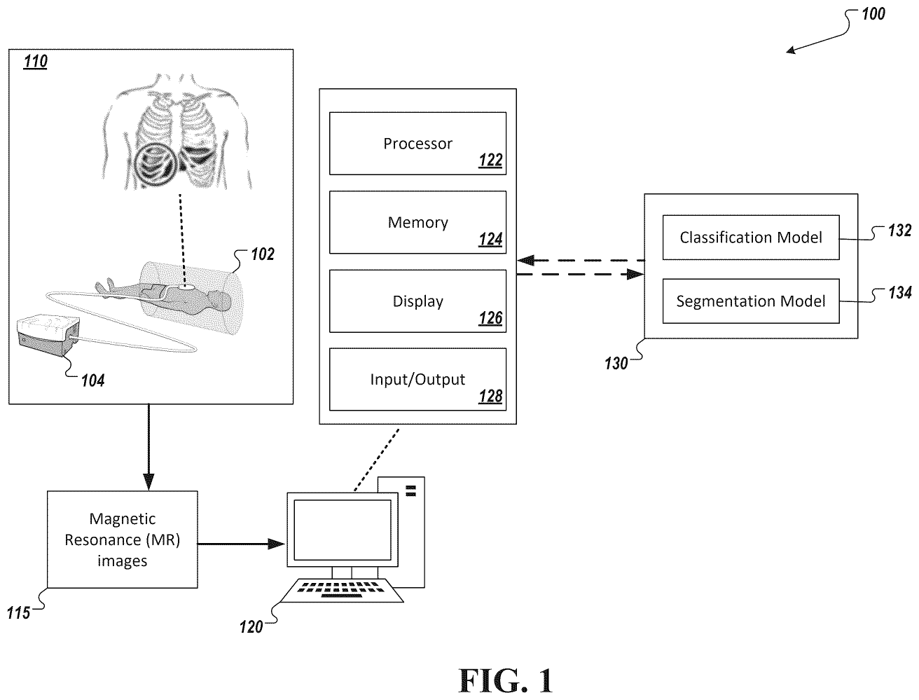

FIG. 1 shows a diagram of an example system 100 utilizing deep learning for automated magnetic resonance elastography (MRE) quality control and stiffness measurements in accordance with an illustrative embodiment.

System 100 includes a MRE imaging device 110 includes a magnetic resonance (MR) scanner 102 configured to collect MRE imaging data 115 of a subject; and a driver 104 configured to generate a plurality of oscillating shear waves in a tissue of interest of the subject (e.g., brain, breast, blood vessels, heart, liver, kidneys, lungs and skeletal muscle). As illustrated in FIG. 1, the driver 104 is positionable about a region of interest (e.g., proximate to the subject's liver) such that the generated oscillations may be recorded within a viewing window of the MR scanner 102.

Typically, the driver 104 is configured to operate at a frequency from 10 Hz to 5000 Hz, such as from 10 Hz to 2500 Hz, from 10 Hz to 1000 Hz, from 10 Hz to 500 Hz, from 50 Hz to 500 Hz, from 10 Hz to 250 Hz, from 50 Hz to 100 Hz, from 50 Hz to 75 Hz, or about 60 Hz. Other general principles of operating a MRE imaging device can be found in Mariappan Y K, Glaser K J, Ehman R L. Magnetic resonance elastography: a review. Clin Anat. 2010 July; 23(5):497-511, which is hereby incorporated by reference in its entirety.

System 100 also includes a computing device 120 including a processor 122; and a memory 124 having instructions 130 stored thereon, wherein execution of the instructions 130 by the processor 122 causes the processor 122 to: obtain magnetic resonance elastography (MRE) imaging data 115 of the subject; determine, via a trained AI classification model 132, a quality assessment metric of the MRE imaging data, wherein the quality assessment metric corresponds to an indication of a diagnostic quality of the MRE imaging data; and screen the MRE imaging data based on the quality assessment metric to provide a filtered data set of MRE imaging data classified as diagnostic quality. Computing device 120 further includes other suitable hardware in accordance with the disclosed subject matter, such as a display 126 and input/outputs 128. In some aspects, the processor 122 can be any suitable hardware processor or combination of processors, such as a central processing unit (CPU), a graphics processing unit (GPU), or a combination thereof.

The AI classification model 132 shown in FIG. 1 uses a SqueezeNet architecture trained using combined inputs of MRE magnitude and the associated 2D FFT by concatenation. The training operation, in the provided example, is configured to employ conventional operations, according to SqueezeNet, e.g., employing gradient descent, loss functions, and various normalization operations, and thus are not further described herein. Other training operations and deep learning (DL) configurations may also be employed.

The system described herein utilizes an AI classification model to determine a quality assessment metric of the MRE imaging data. The term “classification model” refers to a model that uses features of an input (e.g., of an MRE magnitude image) to classify the input into output categories. The classification model can be a binary classification model, which, for example, classifies the input into binary categories such as diagnostic quality and non-diagnostic quality. The associated terms “to classify”, “classifying”, and “performing classification” refer to the operations performed by a classification model. In some aspects, the AI classification model includes a binary classification model. In some aspects, the AI classification model comprises at least one of ResNet18, ResNet34, ResNet50, SqueezeNet, MobileNetV2, or a combination thereof. In some aspects, the AI classification model comprises SqueezeNet. In some aspects, the AI classification model comprises an explainable AI (XAI) model configured to determine, based on features of a non-diagnostic quality image, a predicted artifact source (e.g., magnitude-related artifacts such as presence of an iron deposition, motion artifact, image blurring, and/or poor wave propagation leading to no measurable area despite successful liver delineation). In some aspects, the AI classification model utilizes an ensemble neural network model. The term “ensemble neural network” refers to a neural network that includes one or more sub-networks. The overall inference result from an ensemble neural network may be a weighted combination of the inference result of the individual neural networks in the ensemble.

In some aspects, the MRE imaging data comprises MRE magnitude images, 2D Fast Fourier transform (FFT) of MRE magnitude images, or a combination thereof. In some aspects the MRE imaging data includes confidence map overlaid elastograms (CMOEs). It was advantageously shown in one example that utilizing MRE magnitude slices and/or their 2D FFT counterparts to leverage k-space information instead of CMOEs, resulting in higher average test dataset accuracy (0.919 vs 0.851, respectively). Without wishing to be bound by theory, this improvement may be attributed to the clearer artifact detection and reduced noise from no longer having a hashed pattern in the image.

The system 100 shown in FIG. 1 further includes a trained AI segmentation model 134 configured to generate a segmentation mask corresponding to a region of interest within the filtered data set of MRE imaging data. The term “segmentation model” denotes an unsupervised machine learning model that automatically discovers one or more natural groupings (e.g., “segments”) in data. For example, segmentation models may be used to predict a region corresponding to a target structure (e.g., boundaries of an organ, such as a liver, of a patient) from a medical image (e.g., a MRE magnitude image). In some aspects, the AI segmentation model is configured to only receive diagnostic quality MRE imaging data (e.g., diagnostic quality MRE magnitude images). In various aspects, training of the segmentation model can be accomplished using a training data set including segmentation masks delineated by those skilled in the art or using commercially available software. Other training implementations may also be utilized as would be understood by those skilled in the art.

In some implementations, the trained AI segmentation model comprises an encoder-decoder architecture (e.g., U-Net, ResNet, Multilayer Perceptron, SegNet, Fully Convolutional Networks (FCN), Mask R-CNN, Transformer, Diffusion model, Foundation model, Generative Adversarial Network (GAN), Long short-term memory network (LSTM) or a combination thereof) that takes as input MRE imaging data (e.g., MRE magnitude slices) and returns a segmentation mask. In some examples, the AI model comprises a U-Net architecture.

In some aspects, the trained AI segmentation model is further configured to determine a measurable stiffness area within the filtered data set of MRE imaging data.

In some aspects, the system is further configured to determine the measurable stiffness area for the filtered data set of MRE imaging data using an intersection over union (IoU) of the segmentation mask and a thresholded confidence map obtained from the trained AI segmentation model.

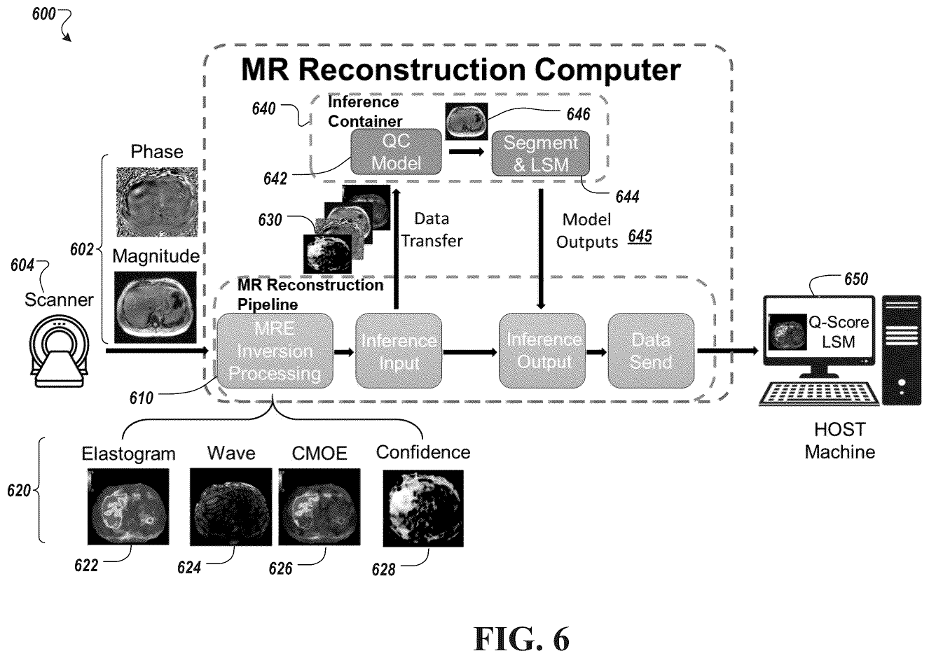

Referring now to FIG. 6, an exemplary system 600 is shown. Briefly, images 602 are collected from a scanner 604 during an MRE procedure. The raw images 602 may be subsequently subjected to inversion algorithms 610 to obtain maps 620 related to mechanical properties of the tissue (e.g., elastograms 622, wave images 624, confidence map overlaid elastograms (CMOEs) 626, and confidence maps 628). Together, this MRE imaging data 630 is provided to an inference container 640 having a quality control (QC) classification model 642 and a segmentation model 644. The QC classification model 642 is configured to receive the MRE imaging data 630 and determine a quality assessment metric, via a trained AI model, denoting whether the MRE imaging data 630 is of diagnostic quality. Using this quality assessment metric, the QC classification model 642 outputs a filtered data set of MRE imaging data 646 to the segmentation model 644. The segmentation model 644 then segments the filtered MRE imaging data 646 and determines a stiffness measurement as an output. The quality assessment metric and the stiffness measurement along with other supporting information is provided as outputs 645 of the inference container 640 before being transmitted to a computing system 650 where clinicians or technical specialists can interpret the results.

### Example Methods

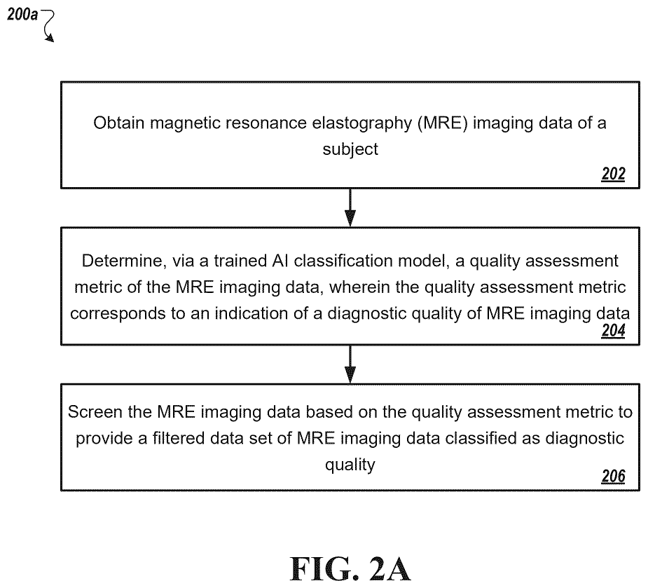

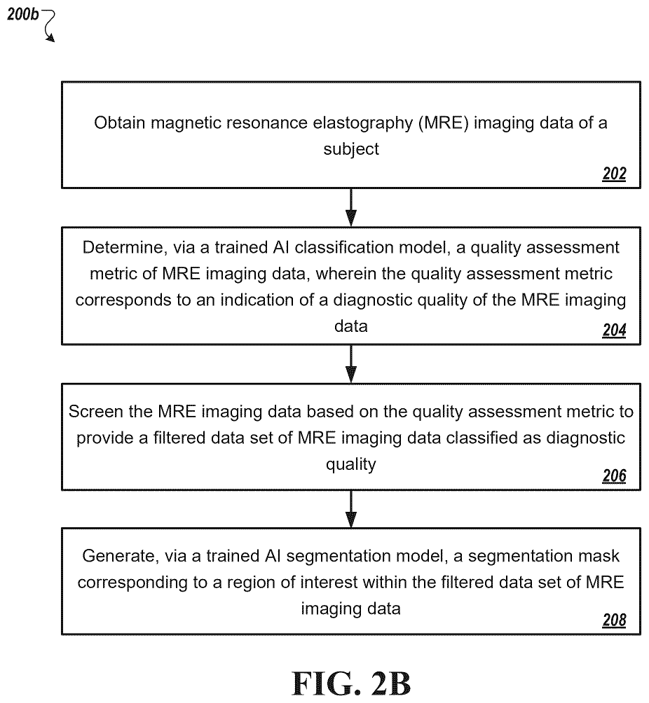

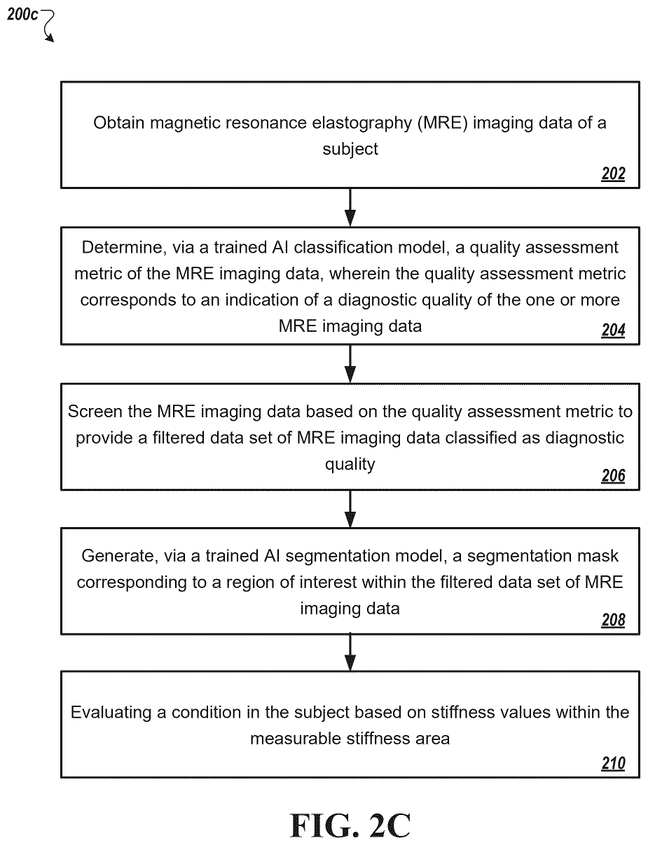

FIGS. 2A, 2B, and 2C each show example methods 200 (shown as 200a, 200b, and 200c) for automated magnetic resonance elastography (MRE) quality control and stiffness measurements in accordance with an illustrative embodiment.

Method 200 includes obtaining (202) magnetic resonance elastography (MRE) imaging data of a subject obtained during a MRE procedure; determining (204), via a trained AI classification model, a quality assessment metric of the MRE imaging data, wherein the quality assessment metric corresponds to an indication of a diagnostic quality of the MRE imaging data; and screening (206) the MRE imaging data based on the quality assessment metric to provide a filtered data set of MRE imaging data classified as diagnostic quality.

In some aspects, the AI classification model comprises a binary classification model. In some aspects, the AI classification model comprises at least one of ResNet18, ResNet34, ResNet50, SqueezeNet, MobileNetV2, or a combination thereof. In some aspects, the AI classification model comprises SqueezeNet. In some aspects, the AI classification model comprises an explainable AI (XAI) model. In some aspects, the XAI model is configured to determine, based on features of a non-diagnostic quality image, a predicted artifact source (e.g., magnitude-related artifacts such as presence of an iron deposition, motion artifact, image blurring, and/or poor wave propagation leading to no measurable area despite successful liver delineation).

In some aspects, the method further includes adjusting a collection of a MRE image (e.g., in real-time) based on the predicted artifact source. In some aspects, the MRE imaging data comprises MRE magnitude images, 2D Fast Fourier transform (FFT) of MRE magnitude images, or a combination thereof.

Referring now to FIGS. 2B and 2C, method 200b, 200c further includes generating (208), via a trained AI segmentation model, a segmentation mask corresponding to a region of interest within the filtered data set of MRE imaging data.

In some aspects, the segmentation mask is subsequently used to determine a measurable stiffness area within the filtered data set of MRE imaging data. In some aspects, the measurable stiffness area is determined for the filtered data set of MRE imaging data using an intersection over union (IoU) of the segmentation mask and a thresholded confidence map obtained from the trained AI segmentation model.

Referring specifically to FIG. 2C, method 200c further includes evaluating (210) or diagnosing a condition in the subject based on stiffness values within the measurable stiffness area. In some aspects, the condition is one or more of cancer, inflammation, and/or fibrosis. In some aspects, the condition is fibrosis. As used herein, the term “fibrosis” refers to the formation of fibrous tissue as a reparative or reactive process, rather than as a normal constituent of an organ or tissue. Fibrosis is characterized by fibroblast accumulation and collagen deposition in excess of normal deposition in any particular tissue. Fibrosis occurs as the result of inflammation, irritation, or healing. The fibrosis may be hepatic/liver fibrosis which references fibrosis occurring in the liver. The method may further be used to evaluate or diagnose cirrhosis of the liver. As used herein, the term “cirrhosis” refers to a late stage of hepatic fibrosis where the liver experiences loss of functional liver cells. Normal liver tissue is replaced with fibrous tissue resulting in widespread distortion of normal hepatic architecture. A major characteristic is regenerative nodules surrounded by dense fibrotic tissue. The overgrowth of fibrosis scar tissue inhibits the liver's proper functioning. Cirrhosis is usually considered irreversible.

As used herein, the term “cancer” includes any form of cancer, including but not limited to, solid tumor cancers such as prostate cancer, breast cancer (e.g., triple-negative breast cancer (TNBC)), brain cancer, ovarian cancer, head and neck cancer, pancreatic cancer, cervical cancer, rectal cancer, esophagus cancer, liver cancer, stomach cancer, testicular cancer, vaginal cancer, uterine cancer, vulvar cancer, paranasal cancer, oropharyngeal cancer, or laryngeal cancer).

Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation.

Exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gout flare, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, type 2 diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopeniaurpura, interstitial cystitis (painful bladder syndrome), gastrointestinal disorder (e.g., selected from peptic ulcers, regional enteritis, diverticulitis, gastrointestinal bleeding, cosinophilic gastrointestinal disorders (e.g., cosinophilic esophagitis, cosinophilic gastritis, cosinophilic gastroenteritis, cosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious ancaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, polymyalgia rheumatic, reperfusion injury, regional enteritis, rheumatic fever, systemic lupus erythematosus, scleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease).

Evaluation/diagnosis of the condition may be achieved by comparing stiffness values determined via the aforementioned methods with normal/abnormal stiffness values. In some aspects, a stiffness value of 2.5 kPa or less is indicative of a normal liver. In some aspects, a stiffness value of 2.93 kPa or more is indicative of a diseased liver.

### Experimental Results and Additional Examples

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers but some errors and deviations should be accounted for. There are numerous variations and combinations of measurement conditions, and other measurement ranges and conditions that can be used to optimize the described process.

A study was conducted to develop and evaluate an automatic deep learning (DL) classifier of liver MRE image quality.

### Experiment #1

The study demonstrated the performance of automated DL-based classification of liver 2D MRE diagnostic quality, with an average accuracy of 0.846 (range 0.719-0.930) across various DL models.

The study was a retrospective, single-center, IRB-approved human study comprising 90 patients (age 52.8±14.1 years, M/F 51/39) using an MRE system having field strengths/sequences 1.5 T and 3 T MRI, 2D GRE, and 2D SE-EPI. The study assessed the curated dataset comprising 914 slices obtained from 149 MRE exams in 90 patients. Two independent observers examined the confidence map overlaid electrograms (CMOEs) for liver stiffness measurement and assigned a quality score (non-diagnostic vs. diagnostic) for each slice. Several DL architectures (ResNet18, ResNet34, ResNet50, SqueezeNet, and MobileNetV2) for binary quality classification of individual CMOE slice inputs were evaluated using an 8-fold stratified cross-validation (800 slices) and a test dataset (114 slices). A majority vote ensemble combining the models' predictions of the highest-performing architecture was evaluated.

The study confirmed, via statistical test, the inter-observer agreement and the agreement between DL models. One observer was assessed using Cohen's unweighted Kappa coefficient. Accuracy, precision, and recall of the cross-validation and the ensemble were calculated for the test dataset.

The study determined that the average accuracy across the 8 models trained using each architecture ranged from 0.692 to 0.846 for the test dataset. The ensemble of the best-performing architecture (SqueezeNet) yielded an accuracy of 0.895. The inter-observer agreement was excellent (Kappa 0.896 [95% CI 0.870-0.922]). The agreement between observer 1 and the predictions of each SqueezeNet model was fair to almost perfect (Kappa range: 0.353-0.856) and substantial for the ensemble (Kappa: 0.780).

Materials and Methods. The study was a retrospective single-center study, approved by the Institutional Review Board with a patient consent exemption. To identify suitable patient records, a query search was conducted using keywords such as “elastography” and “MRE,” within the period from Jan. 1, 2018, to Dec. 31, 2022, utilizing the DICOM database within [Blinded]. From the search, the study selected 149 MRIs, comprising 914 CMOE slices from a total of 90 patients (51 males and 39 females) with a mean age of 52.8±14.1 years and mean BMI of 33.3±8.0 (range: 20.1-51.5) who underwent 2D liver MRE using 1.5 T or 3 T Siemens systems. The selection was based on the clinical reports, aiming to overrepresent failed MRE cases to create a class-balanced dataset for DL training. The inclusion criteria included consecutive patients who underwent MRI and MRE outside a clinical trial or research study, using Siemens systems. The Siemens-based MRE acquisitions generated grayscale CMOEs that were used for training of the DL models uniformly with a single channel input.

MRE Acquisition. MRE exams with 2 different sequences were collected, including a dataset with a 2D spin-echo echo-planar imaging (SE-EPI) sequence acquired at 1.5 T (Magnetom Aera, Siemens Healthineers, n=51) or 3 T (Magnetom Skyra, Siemens Healthineers, n=25); and a dataset with a 2D gradient-echo (GRE) sequence acquired at 1.5 T (Magnetom Aera, n=13) or 3 T (Magnetom Skyra, n=1). Both MRE protocols operated at a vibration frequency of 60 Hz using a Resoundant system (Rochester, MN, USA) (sequence parameters in Table 1).

TABLE 1
2D MRE acquisition parameters obtained on Siemens systems.

| | Scanner | | | |
|---|---|---|---|---|
| | 1.5 T Magnetom Aera | | 3 T Magnetom Skyra | |
| | Sequence Type | | | |
| | SE-EPI | GRE | SE-EPI | GRE |
| Orientation | Axial | Axial | Axial | Axial |
| TR (ms) | 1500, 2000 | 50 | 1000, 1400 | 50 |
| TE (ms) | 41, 48, 52 | 20.7 | 48-50 | 22 |
| Flip angle | 90 | 20, 30 | 90 | 25 |
| (degrees) | | | | |
| FOV (mm²) | 362 × 400- | 407 × 450- | 362 × 400- | 400 × 400 |
| | 453 × 500 | 440 × 440 | 453 × 500 | |
| Matrix | 232 × 256, | 232 × 256, | 232 × 256, | 256 × 128 |
| | 256 × 256 | 256 × 256 | 256 × 256 | |
| Slice thickness | 3, 5, 8 | 10 | 8 | 8 |
| (mm) | | | | |
| Number of slices | 4-10 | 4-8 | 4-6 | 4-8 |
| Acceleration | R = 2 | R = 2 | R = 2 | R = 2 |
| (GRAPPA) | | | | |

Notes:
Comma denotes separate values, hyphen indicates range of values
Abbreviations:
FOV: field of view;
GRE: gradient echo;
MRE: magnetic resonance elastography;
SE-EPI: spin-echo echo-planar imaging;
TE: echo time;
TR: repetition time;
GRAPPA: generalized autocalibrating partially parallel acquisitions

Dataset Processing. Initially, the axial CMOEs were de-identified and preserved in DICOM format. Subsequently, these CMOEs in grayscale format were normalized to a range of [0,255]. Next, they were resized to dimensions of 224×224. Finally, each individual slice was saved as a NumPy array for convenient utilization within the PyTorch framework.

Dataset Labeling. The CMOEs within the entire dataset were labeled based on the percentage of the area of liver parenchyma with 95% or higher confidence, as required in clinical practice (9). An observer (observer 1, XX, a physicist with 2 years of experience in MRE) evaluated each CMOE, and if needed, the other MRE image outputs corresponding to that acquisition, such as magnitude and phase/wave, to determine the quality of the image acquisition as diagnostic or non-diagnostic, as follows: label 0 (non-diagnostic quality, <25% area of confidence in the liver parenchyma), and label 1 (diagnostic quality, ≥25% area of confidence in the liver parenchyma) (9). Another observer (observer 2, YY, a radiologist with 1 year of experience in MRE) reviewed approximately 33% of the dataset for evaluation of inter-observer agreement.

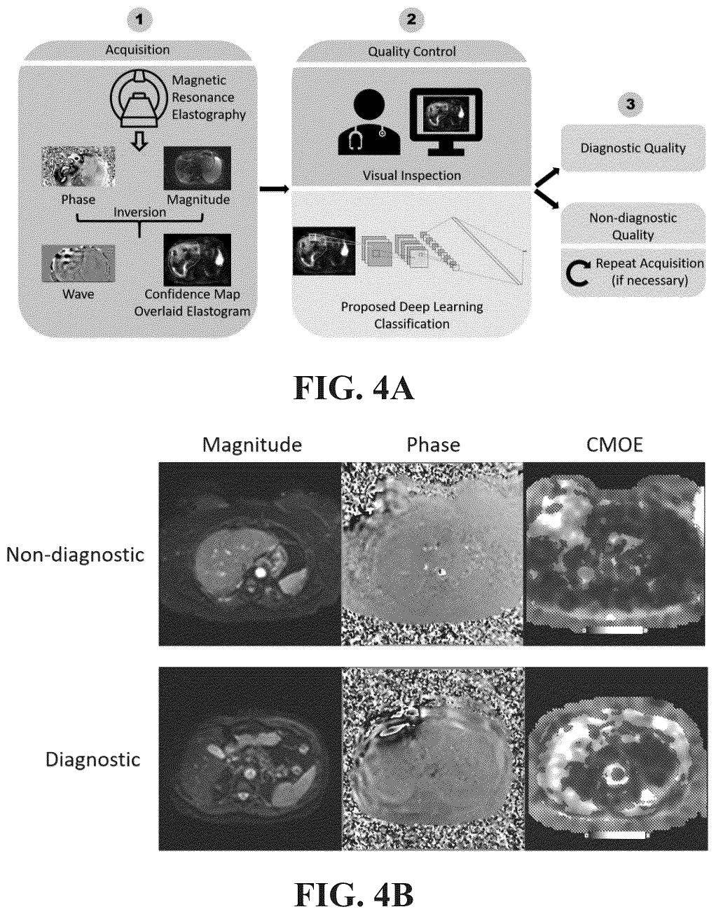

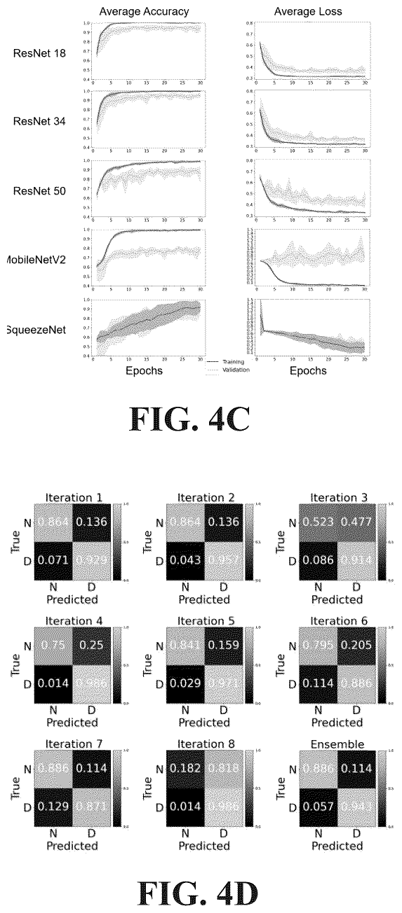

Classification Model. The objective of the instant study was to enhance the MRE quality evaluation process by evaluating various CNNs to obtain a binary classification of CMOEs, determining whether they provided diagnostic or non-diagnostic quality for liver stiffness measurement. The study assessed well-established DL architectures, such as ResNet18, ResNet34, ResNet50, SqueezeNet, and MobileNetV2 in PyTorch (16-21), as shown in FIG. 4A.

FIG. 4A shows a comparison of the workflow between the exemplary DL-based image quality classification and the traditional visual inspection.

The DL various architectures could handle 2D images with dimensions of 224×224 as inputs. Additionally, the study customized the input convolutional layer of each architecture to accept an individual grayscale CMOE slice as input and adjusted the output layer of each architecture for binary classification purposes (shown in FIG. 4B).

FIG. 4B shows examples of curated liver MRE datasets, including a non-diagnostic quality confidence map overlaid elastogram (CMOE) obtained using 2D SE-EPI at 1.5 T (top row) and a diagnostic quality CMOE obtained using 2D SE-EPI at 3 T. The corresponding magnitude and phase images are also shown for each CMOE slice.

TABLE 2
Architecture-specific trainable parameters and average accuracies for the
training, validation, and test datasets. Average precision, recall, and
agreement between each architectural model and reference standard (Cohen's
unweighted Kappa coefficient) also provided for the test dataset.

| | Training | Validation | | | | |
|---|---|---|---|---|---|---|
| | dataset | dataset | | | | |
| Architecture | (n* = 700, | (n* = 100, | | | | |
| (Number of | 76.6%) | 10.9%) | Test dataset (n* = 114, 12.5%) | | | |
| Trainable | Average | Average | Average | Average | Average | Kappa |
| Parameters) | Accuracy | Accuracy | Accuracy | Precision | Recall | Range |
| ResNet18 | 0.998 | 0.944 | 0.798 | 0.839 | 0.594 | [0.304-0.661] |
| (11.2M) | | | | | | |
| ResNet34 | 0.996 | 0.953 | 0.802 | 0.849 | 0.605 | [0.403-0.646] |
| (21.3M) | | | | | | |
| ResNet50 | 0.987 | 0.853 | 0.739 | 0.756 | 0.534 | [0.203-0.760] |
| (23.5M) | | | | | | |
| MobileNetV2 | 0.993 | 0.784 | 0.692 | 0.672 | 0.455 | [0.225-0.393] |
| (2.2M) | | | | | | |
| SqueezeNet | 0.929 | 0.910 | 0.846 | 0.810 | 0.781 | [0.353-0.856] |
| (1.2M) | | | | | | |

Note:
All metrics were calculated after 30 epochs of training. The highest value for each metric is highlighted in bold.
*number of slices

The number of trainable parameters for each architecture was 11.2M (ResNet18), 21.3M (ResNet34), 23.5M (ResNet50), 2.2M (MobileNetV2), 1.2M (SqueezeNet). During the model training, an Adam optimizer was used, along with a cross-entropy loss function to be minimized (referred to as CrossEntropyLoss in PyTorch) (Equation 1) where i∈{0,1} is the class index, tᵢ is a class value either 0 for non-diagnostic quality and 1 for diagnostic quality, and pᵢ is the probability of the respective class outputted by the CNN such that

$${\sum }_{i = 0}^{1} {p}_{i} = 1.$$

$$Loss = - {\sum }_{i = 1}^{2} {t}_{i} log ( {p}_{i} ) \qquad ( Eq . 1 )$$

To verify each architecture's performance and generalizability, an 8-fold stratified cross-validation was performed such that 7 folds, comprising 700 CMOE slices, were used for training and the final fold of 100 slices was used for validation. Each fold maintained the same diagnostic class balance (ratio of diagnostic slices to total number of slices) to mitigate biases during training, based on the reference standard for image quality evaluation reported in the Results. The remaining 114 slices (12.5%) of the dataset were used solely for testing and remained constant across all iterations. Training spanned 30 epochs with a batch size of 32 and a learning rate of 0.0001 (22). Training/evaluation times for the iterations during the cross-validation using an iMac (macOS Ventura 13.2, Processor 3.3 GHZ 6-Core Intel Core i5, Memory 8 GB 2667 MHZ DDR4) are given in Table 3 for each architecture.

TABLE 3
Duration of iterations, test accuracy, and agreement
between each model and reference standard (Cohen's
unweighted Kappa coefficient) using the test dataset.

| | | Duration | Test | Kappa coefficient | |
|---|---|---|---|---|---|
| Architecture | Iteration | (sec) | Accuracy | [95% CI] | |
| ResNet18 | 1 | 2849.27 | 0.816 | 0.596 | [0.516-0.676] |
| | 2 | 2839.30 | 0.789 | 0.524 | [0.437-0.610] |
| | 3 | 2854.78 | 0.781 | 0.510 | [0.424-0.597] |
| | 4 | 2814.98 | 0.842 | 0.661 | [0.588-0.734] |
| | 5 | 2804.54 | 0.842 | 0.649 | [0.573-0.725] |
| | 6 | 2814.92 | 0.711 | 0.304 | [0.202-0.406] |
| | 7 | 2808.40 | 0.807 | 0.579 | [0.498-0.659] |
| | 8 | 2830.60 | 0.798 | 0.554 | [0.470-0.637] |
| | Average | 2827.09 | 0.798 | | |
| ResNet34 | 1 | 5118.22 | 0.789 | 0.524 | [0.437-0.610] |
| | 2 | 5110.68 | 0.825 | 0.607 | [0.527-0.686] |
| | 3 | 5112.63 | 0.798 | 0.546 | [0.461-0.630] |
| | 4 | 5112.18 | 0.833 | 0.631 | [0.554-0.708] |
| | 5 | 5125.35 | 0.719 | 0.403 | [0.313-0.492] |
| | 6 | 5104.48 | 0.798 | 0.541 | [0.456-0.627] |
| | 7 | 5102.54 | 0.816 | 0.596 | [0.516-0.676] |
| | 8 | 5101.98 | 0.842 | 0.646 | [0.569-0.723] |
| | Average | 5111.01 | 0.803 | | |
| ResNet50 | 1 | 8650.14 | 0.728 | 0.382 | [0.287-0.477] |
| | 2 | 8118.78 | 0.667 | 0.203 | [0.097-0.308] |
| | 3 | 8908.69 | 0.798 | 0.580 | [0.501-0.658] |
| | 4 | 7698.00 | 0.667 | 0.171 | [0.061-0.281] |
| | 5 | 7702.88 | 0.746 | 0.400 | [0.304-0.496] |
| | 6 | 7723.27 | 0.711 | 0.342 | [0.246-0.439] |
| | 7 | 7676.90 | 0.711 | 0.456 | [0.376-0.536] |
| | 8 | 7646.30 | 0.886 | 0.760 | [0.698-0.823] |
| | Average | 8015.62 | 0.739 | | |
| MobileNetV2 | 1 | 2641.32 | 0.684 | 0.339 | [0.248-0.43] |
| | 2 | 2639.84 | 0.728 | 0.393 | [0.300-0.486] |
| | 3 | 2563.58 | 0.675 | 0.262 | [0.163-0.362] |
| | 4 | 2530.83 | 0.675 | 0.346 | [0.257-0.434] |
| | 5 | 2539.81 | 0.667 | 0.225 | [0.122-0.327] |
| | 6 | 2514.16 | 0.667 | 0.253 | [0.154-0.352] |
| | 7 | 2530.47 | 0.719 | 0.347 | [0.249-0.445] |
| | 8 | 2559.55 | 0.719 | 0.328 | [0.228-0.429] |
| | Average | 2564.95 | 0.692 | | |
| SqueezeNet | 1 | 2005.91 | 0.825 | 0.620 | [0.543-0.697] |
| | 2 | 1982.75 | 0.912 | 0.815 | [0.759-0.871] |
| | 3 | 1978.57 | 0.737 | 0.405 | [0.311-0.498] |
| | 4 | 2002.15 | 0.930 | 0.856 | [0.806-0.905] |
| | 5 | 2017.14 | 0.895 | 0.780 | [0.72-0.84] |
| | 6 | 2013.20 | 0.719 | 0.353 | [0.256-0.45] |
| | 7 | 2019.58 | 0.860 | 0.723 | [0.658-0.787] |
| | 8 | 2020.20 | 0.895 | 0.782 | [0.722-0.841] |
| | Average | 2004.94 | 0.846 | | |

Note:
The best-performing iteration for each model is highlighted in bold.

Statistical Analysis. The image quality labeling from observer 1 was used as the reference standard for model training and evaluation. Training and validation accuracy (Equation 2) and loss (Equation 1) were calculated for all iterations of the 8-fold cross-validation. The accuracy, precision (Equation 3), and recall (Equation 4) of every trained model were also evaluated using the test dataset. To measure inter-observer agreement and the agreement between the predictions of all DL models and observer 1, Cohen's unweighted Kappa coefficients were calculated (23). Additionally, an ensemble decision-making approach in which the majority vote or mode of the test predictions across all iterations of the highest-performing architecture was evaluated using the test dataset. The same accuracy, precision, recall, and inter-observer agreement metrics were applied to the ensemble for comparison with individual folds.

$$Accuracy = \frac{True Positives + Tru e Negatives}{True Positives + False Positives + True Negatives + False Negatives} \qquad ( Eq . 2 )$$

$$Precision = \frac{True Positives}{True Positives + False Positives} \qquad ( Eq . 3 )$$

$$Recall = \frac{True Positives}{True Positives + False Negatives} \qquad ( Eq . 4 )$$

Results. The Cohen's unweighted Kappa coefficient between the two observers was 0.896 [95% CI0.870-0.922], indicating almost perfect agreement and justifying the use of observer 1's labels. The curated dataset of 914 slices consisted of 574 slices (62.8%) of diagnostic quality and 340 slices (37.2%) of non-diagnostic quality from a total of 149 MRE acquisitions, including 56 failed MRE acquisitions. The technical failure rate was high due to the intentional inclusion of failed MRE datasets for training purposes. The stratified cross-validation maintained a diagnostic class balance of 63.0% (ratio of diagnostic slices to total number of slices) in each of the folds, and the test dataset had a diagnostic class balance of 61.4%. The number of slices acquired using the 1.5 T system was 718 (78.5% of all slices), with 478 slices (66.5% of 1.5 T slices) labeled diagnostic and 240 slices (33.5% of 1.5 T slices) labeled non-diagnostic. Similarly, the number of slices acquired using the 3 T system was 196 (21.5% of all slices), with 96 slices (49.0% of 3 T slices) labeled diagnostic and 100 slices (51.0% of 3 T slices) labeled non-diagnostic. As for the sequences used for the acquisition, 838 slices (91.7% of all slices) were acquired using EPI, with 513 slices (61.2% of EPI slices) labeled as diagnostic and 325 slices (38.8% of EPI slices) labeled as non-diagnostic. A total of 76 slices (8.3% of all slices) were acquired using GRE, where 61 slices (80.3% of GRE slices) were labeled as diagnostic and 15 slices (19.7% of GRE slices) were labeled as non-diagnostic.

DL Model Performance. The training/validation accuracy and loss averages with their standard deviations during the 30 epochs of training across all cross-validation iterations for each architecture are shown in FIG. 4C.

FIG. 4C shows training/validation accuracies and losses for each DL model across cross-validation iterations. For each iteration, the training/validation datasets consisted of 700/100 slices, respectively. The solid line represents the average accuracy or loss across all 8 iterations of the cross-validation, and the band represents the corresponding standard deviation.

Training for 30 epochs yielded optimal test accuracy performances, with little to no improvement occurring at higher epochs, and overfitting increasing at 35 epochs, except for the SqueezeNet model. The average accuracies ranged from 0.692 to 0.846 using the test set (Table 2). The average training, validation, and test accuracies, as well as test precision and test recall calculated from each trained model during the cross-validation, are shown in Table 3. The highest average test precision (0.849) was achieved across the ResNet50 models, while the highest average accuracy (0.846) and average recall (0.781) were achieved across the SqueezeNet models. Therefore, the study considered SqueezeNet to be the highest-performing architecture.

Confusion matrices of each cross-validation iteration of the SqueezeNet are included to show the class-specific predictions versus observer labeling, as shown in FIG. 4D.

FIG. 4D shows confusion matrices for each cross-validation iteration, where (N) signifies non-diagnostic image quality and (D) signifies diagnostic image quality, using the test dataset (114 slices). The matrices were generated for the best-performing architecture, SqueezeNet, as well as its corresponding ensemble decision-making approach. The color gradient represents the % of images of each true class that were predicted to be N or D. A perfect classification would be represented by 1.0 in the main diagonal (top-left and bottom-right) of a confusion matrix.

The ensemble decision-making approach for the SqueezeNet achieved an accuracy of 0.895, a precision of 0.926, and a recall of 0.900 in the test set. Lastly, the SqueezeNet models trained in the shortest amount of time (average: 2004 seconds) compared to the models using other architectures (range of averages: 2564-8015 seconds) (Table 3).

Agreement Between Models and Reference Standard. The models using the SqueezeNet architecture achieved varying levels of agreement with the reference standard, including fair (0.21-0.40) in 2 iterations, substantial (0.61-0.80) in 4 iterations, almost perfect (0.81-1.00) in 2 and substantial in the ensemble decision-making approach (Cohen's unweighted Kappa 0.780 [95% CI 0.72-0.84]) (Table 3). Among the other architectures, the highest Kappa coefficient between the reference standard and the model was achieved at the 8th iteration of the Resnet50 cross-validation (Kappa: 0.760, accuracy: 0.886). For the lowest performing architecture, the MobileNet, the 2^{nd} iteration was the best iteration, which yielded a Kappa of 0.393 and an accuracy of 0.728.

Discussion. The study assessed the performance of various established DL architectures for classifying liver MRE quality using annotated CMOE slices. The study found that SqueezeNet models yielded the highest average accuracy (0.846), average recall (0.781), and agreement with the reference standard (average Cohen's unweighted Kappa coefficient of 0.667) using the test dataset. Additionally, the study observed that the average training duration across the SqueezeNet models (2004 seconds) was faster than the average training duration of the models that used other architectures (range of averages: 2564-8015 seconds). Automating MRE quality review may enable technicians and/or radiologists to focus on correcting the quality issues, such as adjusting hardware and correcting patient motion, so that reacquisitions may be performed as required within the session's time constraints. No previous studies have reported a DL-based automated quality control of 2D liver MRE using confidence maps overlaid with elastograms.

The study considered each slice from an MRE exam as an input, rather than the entire dataset of the MRE exam. This approach was favorable for increasing the sample size of the dataset. In addition, slices from repeated MREs from the same patient could be used in the dataset since different MRE in the same patient could yield different quality outcomes. To accommodate this, data from the same patients might have appeared in multiple folds during the stratified cross-validation, as each CMOE slice was evaluated individually rather than giving a single score to a complete exam. An alternative approach would have required a 3D input, with a much larger sample size, and potentially different CNN architectures, which could be the subject of future studies. Given these considerations, the study did not expect a major bias to affect the model evaluation based on the potential of data from a single acquisition being present in both the training and validation cohorts.

On average, substantial agreement was observed between the best-performing DL model (SqueezeNet architecture) and the reference standard, with an average Cohen's unweighted Kappa coefficient of 0.667, up to 0.856 (in its 4^{th} iteration). Although slightly lower than the inter-observer agreement (Kappa 0.896), it surpassed the substantial inter-observer agreement reported in a previous study by Wagner et al (Kappa=0.781) (9). Our approach establishes a baseline for automating MRE quality control using a well-established CNN architecture (SqueezeNet) and achieving an average test accuracy of 0.846, with a peak test accuracy of 0.930 in its best-performing iteration.

Comparing the performance of various CNN architectures (ResNet-18, ResNet-34, ResNet-50, SqueezeNet, and MobileNetV2), it is noteworthy that despite having a lower count of trainable parameters, SqueezeNet exhibited the best performance in our study. Specifically, SqueezeNet achieved superior results in terms of accuracy, recall, and Cohen's unweighted Kappa coefficient for the test dataset compared to the other architectures upon training. The superior performance of SqueezeNet despite its lower count of trainable parameters could be attributed to its unique architecture. SqueezeNet introduces several innovative design choices, such as replacing the traditional large filter size with a combination of 1×1 and 3×3 filters, reducing the parameters without compromising performance. Furthermore, the lower number of trainable parameters (1.2M) of the SqueezeNet is likely to be more favorable for the smaller-scale dataset used in this study. Yet, a lightweight architecture does not necessarily guarantee good performance, as demonstrated by the MobileNetV2 (2.2M trainable parameters), which yielded the lowest average test set accuracy (0.692) of the tested architectures.

The study may investigate the impact of including additional MRE output images, such as magnitude and phase images, as inputs to the classification models for enhancing prediction accuracy.

### Experiment #2

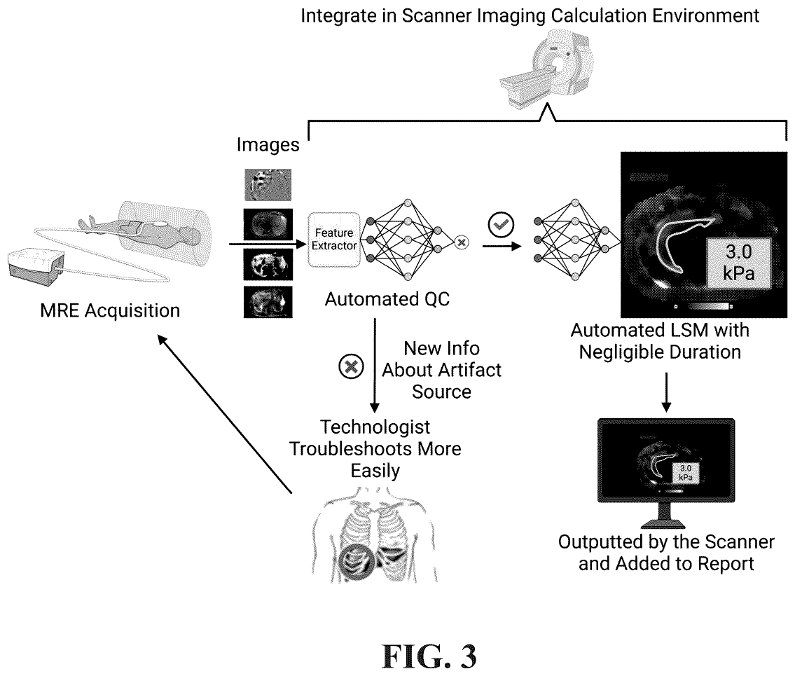

The study also conducted a second experiment to demonstrate the performance of automated DL-based classification of liver 2D MRE diagnostic quality. A general overview of the system architecture is shown in FIG. 3.

Materials and Methods. This experiment of the study covered the period from Jan. 1, 2018, to Dec. 31, 2022, and utilized a DICOM database, yielding 1,372 MRE exams. The inclusion criteria for the MRE exams were: (1) patients who had MRI and 2D MRE using Siemens scanners outside of a clinical trial, and (2) availability of grayscale elastogram outputs. The exclusion criterion for the MRE exams was: MRE data from non-Siemens vendors, such as GE systems. From these exams, the study randomly selected 897 MRE magnitude slices along with the other MRE outputs from 146 MRE acquisitions from 69 patients (37 males; age 51.6±14.8 years; BMI 32.5±7.7, Table 4) who underwent 2D liver MRE using 2 different sequences, 2D spin-echo echo-planar imaging (SE-EPI) at 1.5 T (Magnetom Aera, Siemens Healthineers, n=629 slices) or 3 T (Magnetom Skyra, Siemens Healthineers, n=194 slices); and 2D Gradient Echo (GRE) at 1.5 T (Magnetom Aera, Siemens Healthineers, n=74 slices). The study chose a dataset of close to 900 images for DL model training, balancing the need for sufficient training data with the practical limitations of manual annotation. To achieve this, the study randomly selected patients to curate a representative and manageable dataset. The study made a choice of a single vendor (Siemens Healthineers) to streamline image pre-processing and restrict the dataset to grayscale images. The overrepresentation of failed MRE slices was deliberate to provide a balanced class distribution in this curated dataset. While confidence map overlaid elastograms (CMOEs) were previously used for QC [15′], the instant study instead used the MRE magnitude slices, their Fast Fourier transform (FFT) counterparts, as well as confidence maps and elastograms of the same dataset.

TABLE 4
Clinical characteristics of the patient cohort (n = 69) in the study.

| | | |
|---|---|---|
| Age (years) mean ± SD (range) | 51.6 ± 14.8 | (22-77) |

| | |
|---|---|
| Gender (M/F) | 37/32 |

Etiology of chronic liver disease

| MASLD/MASH | n = 43 | |
|---|---|---|
| Autoimmune hepatitis | n = 1 | |
| Primary sclerosing cholangitis (PSC) | n = 1 | |
| Alcohol use disorder | n = 5 | |
| HBV | n = 4 | |
| HCV | n = 4 | |
| Primary biliary cholangitis (PBC) | n = 2 | |
| Other (Genetic hemochromatosis, | n = 9 | |
| Cryptogenic cirrhosis, Congestive | | |
| hepatopathy, Biliary atresia, Unknown) | | |
| Body mass index (BMI)* | 32.5 ± 7.7 | (20.1-51.5) |

Ethnicity

| | |
|---|---|
| Asian | n = 7 |
| Black or African American | n = 8 |
| Hispanic or Latino | n = 6 |
| White | n = 28 |
| Other/Unknown | n = 20 |

Note:
*Not available in 2 patients.
Abbreviations: MASLD: metabolic dysfunction-associated steatotic liver disease; MASH: metabolic dysfunction-associated steatohepatitis.

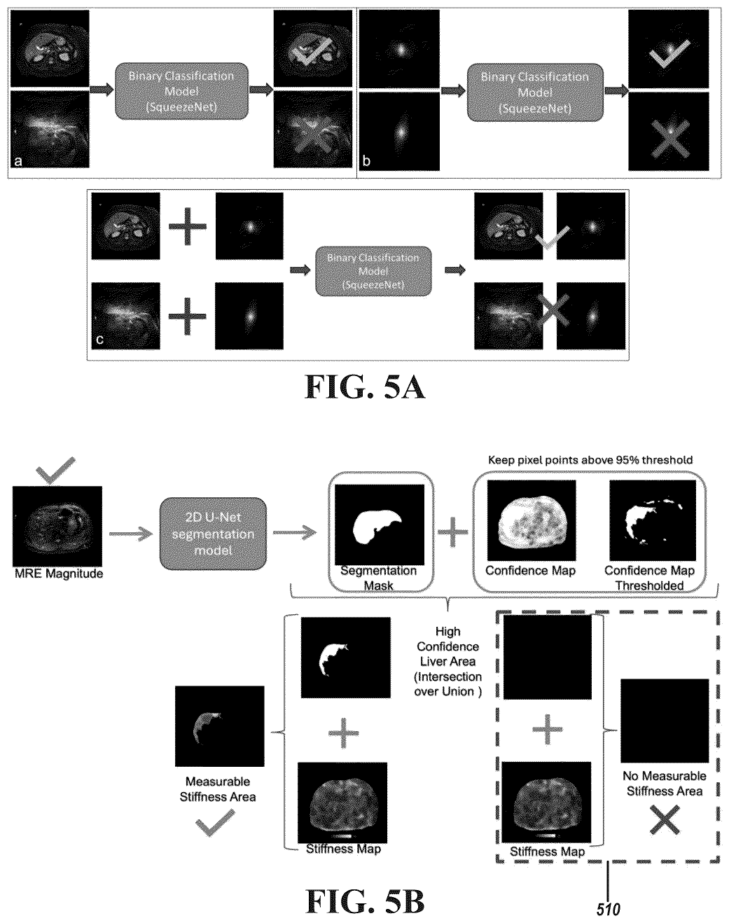

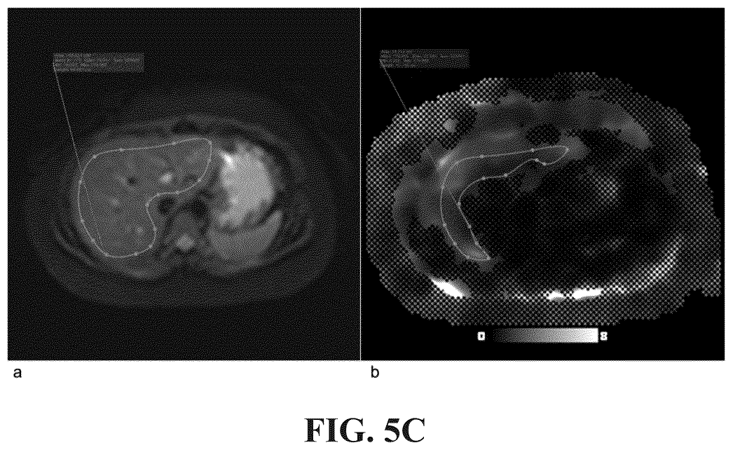

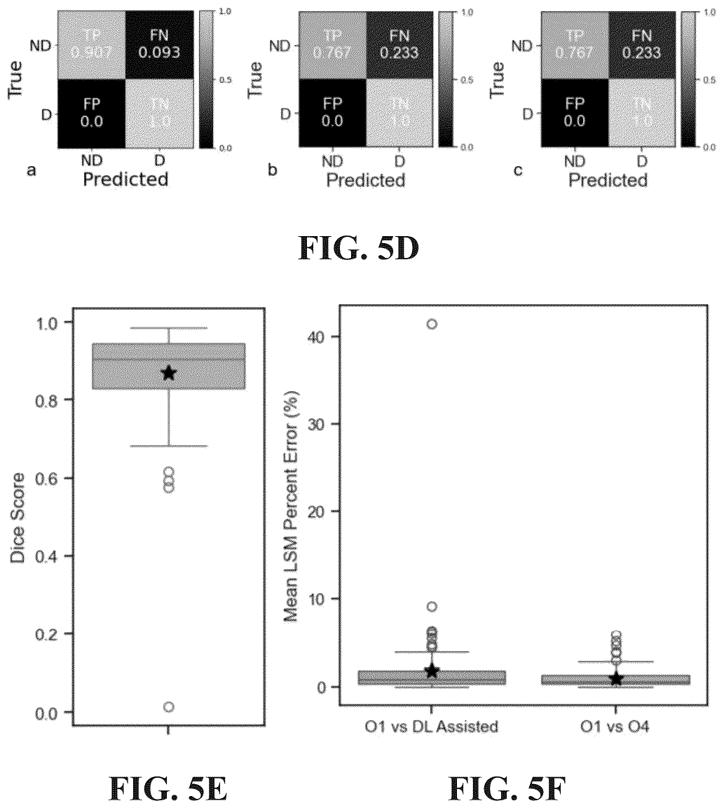

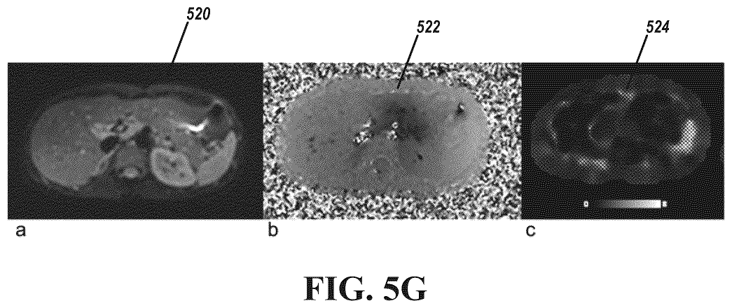

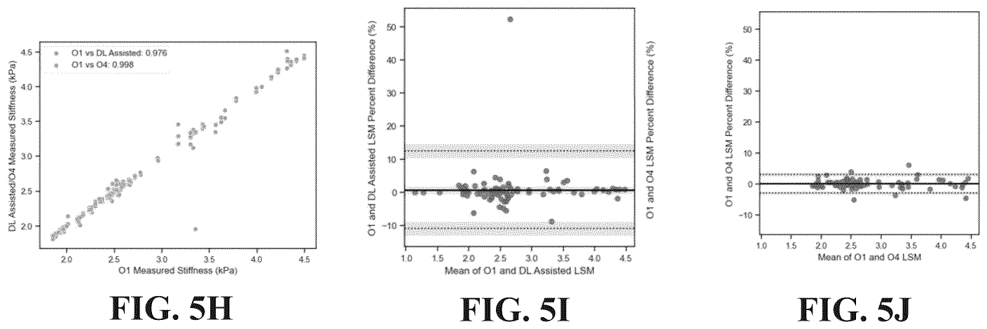

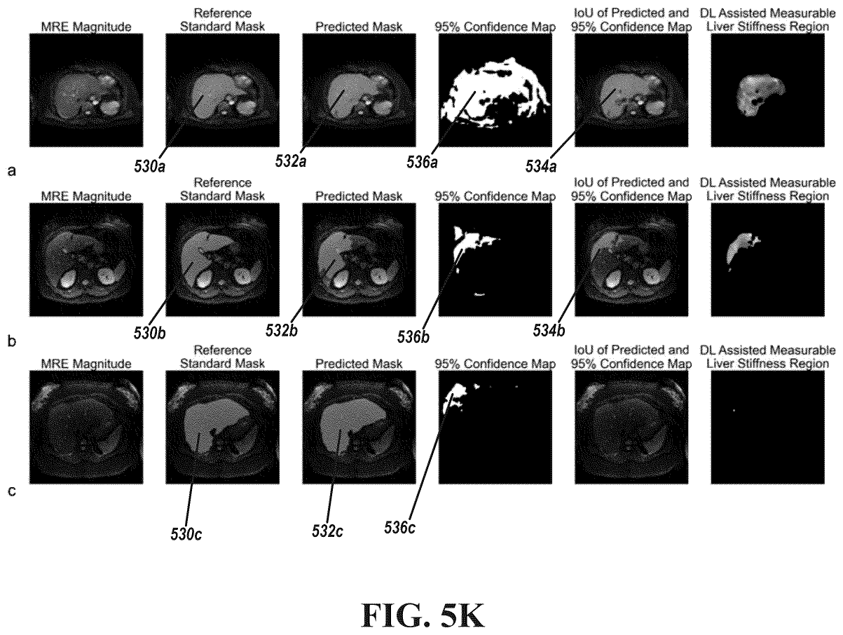

Deep Learning (DL) Model. The exemplary system and method (also referred to as DL-assisted system and method herein) can comprise two portions/steps, each employing a same or different DL model (e.g., classification, segmentation). FIG. 5A shows a binary classification model (e.g., SqueezeNet), in the first portion/step of the exemplary system, configured for automated quality control (QC) assessment. MRE magnitude slices classified as of diagnostic quality by the binary classification model can be used for subsequent DL-assisted liver stiffness measurement by a segmentation model (FIG. 5B). As shown, the binary classification model can accept MRE magnitude images (subpanel (a)), two-dimensional (2D) Fast Fourier Transform (FFT) images, or MRE magnitude images combined with 2D FFT images (subpanel (c)) as inputs.

FIG. 5B shows a segmentation model (e.g., 2D U-Net segmentation model), in the second portion/step of the exemplary system, configured for measurable liver area delineation and image overlaying steps to perform DL-assisted liver stiffness measurement on the MRE magnitude images (or slices) classified as diagnostic quality by the binary classification model (FIG. 5A). Box 510 shows an example in the case of a low confidence map output that results in no measurable stiffness area to be present. Regardless of the high confidence map coverage, the exemplary system (i.e., pipeline) outputted a stiffness value in kPa in the experiment.

To enhance the MRE review process, the study implemented a two-step explainable quality control (QC) system and method with subsequent liver stiffness measurement (LSM) output for diagnostic quality exams. In the first portion/step of the system, a DL binary classification model (shown in FIG. 5A) assesses MRE magnitude images to detect visual artifacts such as patient motion-related, aliasing, or blurring. If the magnitude images pass the quality check at the DL binary classification model, the images can be transferred to a liver segmentation model (shown in FIG. 5B), in the second portion/step of the system, which outlines liver boundaries to assist LSM. Then, the intersection over union of the predicted segmentation and the binary confidence mask can be calculated to obtain the measurable area within the liver. If there is no measurable area (the second QC step, shown in box 510), then there is a lack of a high signal-to-ratio (SNR) displacement region in the confidence map due to poor or no shear wave propagation, despite the segmentation model delineating the liver.

In doing so, the exemplary system and method can yield two outcomes following an MRE exam: (i) diagnostic quality results with LSM, or (ii) non-diagnostic quality results, either due to poor MRE magnitude quality preventing liver delineation, or poor wave propagation leading to no measurable area despite successful liver delineation, aiding technologists in troubleshooting.

Data Labeling. Three independent observers, observer 1 (MY), 2 (KY), and 3 (EA), evaluated all the MRE magnitude slices to determine their diagnostic quality. Slices were labeled as diagnostic (1) or non-diagnostic (0) quality by each observer based on their suitability for liver delineation, which can be affected by the presence of iron deposition, motion artifact, or image blurring. The reference standard for each slice was based on the majority vote/highest number of votes among the 3 observers.

Additionally, observers 1 and 4 (EO) independently delineated the liver regions to create binary masks from the MRE magnitude slices for LSM. Observer 1 used software (3D Slicer version 4.11) to provide liver masks as the reference standard for training the segmentation model, while Observer 4 used ImageJ to acquire liver masks for interobserver difference analysis (in all the datasets). Software selection was based on user preference and familiarity to complete the liver segmentation process as efficiently as possible. All the diagnostic quality MRE magnitude slices, as determined based on the reference standard procedure described herein, and the segmentation masks delineated by Observer 1, were utilized to train the 2D U-Net-based segmentation model.

Data Processing. The axial MRE magnitude, stiffness/elastogram, and confidence map slices were anonymized and saved in DICOM format, with each slice also stored as a NumPy array for easy integration with the PyTorch framework. Only the MRE magnitude slices underwent normalization and standardization to a [0, 255] range. Subsequently, all images were resized to dimensions of 224×224, and the corresponding 2D FFTs of the MRE magnitude images were computed. On the other hand, the segmentation masks were stored as JPEG files and converted to NumPy arrays during model training. Confidence map slices were thresholded at 95% confidence for binarization.

Classification and Segmentation Models. For binary classification (shown in FIG. 5A) in the first portion/step of the exemplary system, the SqueezeNet model was used with a customized input convolutional layer to accommodate combined inputs of MRE magnitude and the associated 2D FFT by concatenation. Additionally, the same SqueezeNet model was adjusted to accommodate a single-channel input to individually evaluate the performance of MRE magnitude and 2D FFT slices. The SqueezeNet was selected due to its effective balance between performance and model size [15′]. During model training, an NVIDIA Geforce RTX 3050 was used with CUDA v12.4. An Adam optimizer and a cross-entropy loss function were employed. An eightfold stratified cross-validation was performed, with 7 folds (667 MRE magnitude slices, 75%) used for training and the final fold (95 slices, 10%) used for validation. The remaining 135 slices (15%) were reserved for testing and remained constant across all iterations. The training was conducted over 50 epochs with a batch size of 32 and a learning rate of 0.0001. Predictions from each iteration were combined into a majority vote decision on slice quality.

For the segmentation (e.g., liver ROI selection) and DL-assisted LSM tasks (shown in FIG. 5B) in the second portion/step of the exemplary system, only the diagnostic quality MRE magnitude slices (615 slices), which 70% (430 slices) were used for training, 15% (92 slices) were used for validation, and 15% (93 slices) reserved for the test dataset, were inputted into a segmentation model based on the widely used 2D U-Net architecture [17′] that built upon an encoder-decoder structure, along with their corresponding reference standard liver masks. The training was allowed to run for up to 150 epochs with a batch size of 16, a learning rate of 0.0001, and a Dice loss function. Early stopping was enabled to end training after 10 epochs of no improvement in the loss function applied to the validation dataset.

The 2D U-Net model's predicted liver masks in the second portion/step were multiplied elementwise with confidence maps (thresholded at a 95% confidence level) to generate final binary masks, denoting high-confidence measurable areas within the liver. This procedure focused on the first MRE magnitude slice out of 4 collected for each confidence map and stiffness map, wherein each slice corresponded to one of four phases of data collected over time, with the first MRE magnitude slice chosen from the initial time point to perform LSM.

Efficiency and Statistical Analyses. An LSM efficiency evaluation was performed to compare the duration of manual LSM vs DL-assisted LSM using 4 separate MRE acquisitions, totaling 29 slices, obtained from the same query search. FIG. 5C shows a polygon tool used by an observer for LSM efficiency evaluation. In subpanel (a), for each diagnostic MRE magnitude slice, observer 1 used the polygon tool (e.g., Horos Project) to draw ROIs. In subpanel (b), these ROIs were then transferred onto the CMOEs to outline liver regions with adjustments to exclude hashed areas (lower than 95% confidence level). The duration of the manual LSM was recorded.

The study also compared the performance of the DL-assisted QC and LSM methods (shown in FIGS. 5A-5B) against the manual approach (the reference standard). For the binary QC classification models, accuracy, precision, recall, and F1-score (harmonic mean of precision and recall) were computed for the test dataset. Predictions from all iterations were ensembled to enhance the robustness of the DL-based classification. Cohen's Kappa coefficient was utilized to evaluate the agreement between the DL-based predictions and the labels assigned by the observer's majority vote scores. The Dice score was calculated to assess the spatial overlap between the liver segmentations produced by the 2D U-Net model and the reference standard liver masks by observer 1. Additionally, the mean LSM error (in %), defined per Equation 5, was calculated to compare the manual measurements (i.e., referenced standard) performed by Observer 1 against the DL-assisted approach and those obtained by Observer 4. The intraclass correlation coefficient (ICC) was also computed to assess the level of agreement between Observer 1's LSMs and those provided by the DL-assisted approach and Observer 4's, along with Bland-Altman limits of agreement. In the efficiency evaluation, the duration of manual LSM by Observer 1 was compared against the duration of the DL-assisted approach using a paired t-test, in which slices were grouped per patient.

$$Mean LSM Percent Error = \frac{\vert LSM - Reference Standard LSM \vert}{Reference Standard LSM} \times 100 \% \qquad ( Eq . 5 )$$

Deep Learning Model Performance Results. The dataset labeled using a majority-vote approach resulted in 615 slices being classified as diagnostic and 282 as non-diagnostic, which served as the quality control reference standard. Out of the total 897 slices, 667 were used for training, 95 for validation, and 135 for testing. The majority vote of the observers, used as the reference standard, showed the following Cohen's Kappa coefficient when compared to each individual observer: 0.930 [95% CI 0.904-0.956] with Observer 1, 0.890 [95% CI 0.857-0.922] with Observer 2, and 0.916 [95% CI 0.889-0.945] with Observer 3. All 615 diagnostic slices were segmented by Observer 1 to measure the slice-specific liver stiffness (in kPa) and were used for segmentation model training and evaluation. Sample slices from the diagnostic and non-diagnostic groups are shown in FIG. 5A.

A single-channel DL model, which utilized only MRE magnitude slices as input, achieved average accuracy, precision, recall, and F1-score of 0.950 [range: 0.897-0.970], 0.967 [range: 0.774-1.000], 0.881 [range: 0.791-0.977], and 0.918 [range: 0.854-0.955], respectively, on the test dataset (136 slices). Cohen's Kappa coefficients, comparing the DL model's predictions to the reference standard (the majority vote of the observers), ranged between 0.776 and 0.933, indicating the single-channel DL model was the best-performing binary classification model for QC. Results for FFT-only models and MRE magnitude combined with FFT models were provided in Table 5 and Table 6. FIG. 5D shows confusion matrices for DL models using only MRE magnitude images, only 2D FFT images, and a combination of MRE magnitude and 2D FFT images. These matrices compared the ensemble predictions of the DL-assisted QC model to the reference standard.

TABLE 5
Performance of the SqueezeNet models for MRE magnitude image quality control

| | Training | Validation | | | | | |
|---|---|---|---|---|---|---|---|
| | dataset | dataset | | | | | |
| Input type | (n = 667, | (n = 95, | | | | | |
| (number of | 75%) | 10%) | Test dataset (n = 135, 15%) | | | | |
| trainable | Average | Average | Average | Average | Average | Average | Kappa |
| parameters) | accuracy | accuracy | accuracy | precision | recall | F1 score | range |
| MRE | 0.943 | 0.912 | 0.950 | 0.967 | 0.881 | 0.918 | 0.776- |
| magnitude | | | | | | | 0.933 |
| (727K) | | | | | | | |
| 2D FFT of | 0.972 | 0.945 | 0.919 | 0.991 | 0.752 | 0.854 | 0.759- |
| MRE | | | | | | | 0.838 |
| magnitude | | | | | | | |
| (727K) | | | | | | | |
| MRE | 0.959 | 0.911 | 0.919 | 0.997 | 0.747 | 0.853 | 0.698- |
| magnitude | | | | | | | 0.857 |
| combined | | | | | | | |
| with 2D | | | | | | | |
| FFT (731K) | | | | | | | |

Note:
The percentage of diagnostic slice count was fixed at 68% across the training, validation, and test datasets. Average accuracies specific to input type for training, validation, and test datasets, along with average precision, recall, F1-score, and the range of Cohen's Kappa, agreed with the reference standard across the cross-validation iterations on the test dataset. All metrics were calculated after 50 epochs of training. The highest value for each metric is highlighted in bold. n is the number of slices.

TABLE 6
Cross-validation performance of the SqueezeNet
models for MRE magnitude image quality control.

| Input Type | Iteration | Accuracy | F1 Score | Kappa coefficient |
|---|---|---|---|---|
| MRE | 1 | 0.971 | 0.951 | 0.930 |
| magnitude | 2 | 0.971 | 0.955 | 0.933 |
| | 3 | 0.956 | 0.925 | 0.894 |
| | 4 | 0.949 | 0.911 | 0.876 |
| | 5 | 0.897 | 0.854 | 0.776 |
| | 6 | 0.963 | 0.938 | 0.912 |
| | 7 | 0.934 | 0.883 | 0.838 |
| | 8 | 0.941 | 0.897 | 0.857 |
| | Average | 0.950 | 0.918 | |
| | Ensemble | 0.971 | 0.951 | 0.930 |
| 2D FFT of | 1 | 0.926 | 0.868 | 0.819 |
| MRE | 2 | 0.912 | 0.838 | 0.779 |
| magnitude | 3 | 0.912 | 0.850 | 0.788 |
| | 4 | 0.919 | 0.853 | 0.799 |
| | 5 | 0.926 | 0.868 | 0.819 |
| | 6 | 0.904 | 0.822 | 0.759 |
| | 7 | 0.912 | 0.838 | 0.779 |
| | 8 | 0.934 | 0.883 | 0.838 |
| | Average | 0.919 | 0.854 | |
| | Ensemble | 0.926 | 0.868 | 0.819 |
| MRE | 1 | 0.912 | 0.842 | 0.782 |
| magnitude | 2 | 0.919 | 0.853 | 0.799 |
| combined with | 3 | 0.919 | 0.853 | 0.799 |
| 2D FFT | 4 | 0.934 | 0.883 | 0.838 |
| | 5 | 0.882 | 0.771 | 0.698 |
| | 6 | 0.912 | 0.838 | 0.779 |
| | 7 | 0.926 | 0.868 | 0.819 |
| | 8 | 0.941 | 0.897 | 0.857 |
| | Average | 0.919 | 0.853 | |
| | Ensemble | 0.926 | 0.868 | 0.819 |

Note:
Accuracy, F1 score, and Cohen's Kappa coefficient for each cross-validation iteration compared the classification predictions of the test dataset with respect to the reference standard.

Automated Processing of LSM. FIG. 5E shows Dice scores between the segmentation masks delineated by the predicted masks of a DL segmentation model (e.g., 2D U-Net) and the reference standard (Observer 1), demonstrating the median (Q2) of 0.90, interquartile range (IQR=Q3−Q1) of 0.12, and mean (indicated by a star) of 0.87 for 93 diagnostic quality slices in the test dataset. FIG. 5F shows the distributions of the liver stiffness measurement (LSM) error (in %), comparing the deep learning (DL)-assisted method (e.g., 2D U-Net segmentation model) for diagnostic-quality slices and the reference standard (Observer 1) and between Observers 1 and 4, in the test dataset, with median, IQR, and mean as follows: DL segmentation model (e.g., 2D U-Net) vs Observer 1 (O1): 0.85, 1.50, 1.90; and Observer 1 (O1) vs. observer 4 (O4): 0.58, 1.06, 1.00 (for n=82 slices).

The early stopping ended training after 82 epochs for the 2D U-Net-based segmentation model, which achieved a Dice score of 0.87 on the test dataset (shown in FIG. 5E). The DL-outputted segmentation masks, along with the MRE-outputted confidence maps and the elastograms/stiffness maps, were used for LSM shown in FIG. 5B.

FIG. 5G shows an MRE example slice demonstrating a diagnostic-quality MRE magnitude slice 520, its associated phase slice 522, and the confidence map overlaid with the elastogram slice 524.

Although the 93 MRE magnitude slices were of diagnostic quality, 5 of their corresponding confidence maps did not result in high-confidence measurable liver areas (shown in FIG. 5A, subpanel (b); and FIG. 5G). Consequently, despite the segmentation masks being created by Observer 1 and the DL-assisted method to outline the liver area, these 5 slices were excluded from the LSM analysis due to the lack of a high-confidence measurable area. These cases failed the second step of the QC process, indicating poor or no shear wave propagation since the corresponding MRE magnitude images were classified as diagnostic quality in the first QC step. The mean LSM percent error between the DL-assisted method and the reference standard (Observer 1) was 1.9%+4.6% (shown in FIG. 5F) for 88 slices in the segmentation-specific test dataset.

FIG. 5H shows a scatterplot comparing liver stiffness measurements (LSMs) between the deep learning (DL)-assisted method and Observer 1 (n=88 diagnostic quality slices), and between Observer 1 (O1) and Observer 4 (O4) (n=82 diagnostic quality slices) with intraclass correlation (ICC). FIG. 5I shows a Bland-Altman plot for the DL-assisted method vs. O1 with a mean LSM difference of 0.79% [1.96 SD=12.54 and—1.96 SD=−10.97]. FIG. 5J shows a Bland-Altman plot for O1 vs O4 with a mean LSM difference of 0.09% [1.96 SD-3.13 and—1.96 SD=−2.96].

In FIG. 5H, the ICC calculation for LSMs was performed on these 88 slices between the DL-assisted method and Observer 1, yielding 0.976 (p<0.0001). Observer 4 could not delineate the liver region on the MRE magnitude images for 6 slices of the 88, causing the LSMs for these slices not to be reported. The ICC calculated for LSMs performed on 82 slices by observers 1 and 4 was 0.998 (p<0.0001), while the mean LSM error was 1.0±1.2% (shown in FIG. 5F).

The differences in LSMs between the DL-assisted approach and Observer 1 and between Observers 1 and 4, were reported as Bland-Altman plots in FIGS. 5I-5J, showing mean percent difference of 0.79% [1.96 SD=12.54 and—1.96 SD=−10.97] and 0.09% [1.96 SD=3.13 and—1.96 SD=−2.96], respectively. FIG. 5K shows examples of the segmentation and overlaying procedure that can be used to obtain automated stiffness measurements.

In FIG. 5K, subpanel (a) shows a high Dice score (0.95) segmentation and good confidence coverage with predicted measurable liver area (91%). Subpanel (b) shows a moderate Dice score (0.84) segmentation with moderate confidence coverage of the predicted measurable liver area (38%). Subpanel (c) shows a high Dice score (0.97) segmentation with no high-confidence measurable liver area (0.1%).

In subpanels (a)-(c), mask 530 (shown as 530a-530c) (column 2) is the true label delineating the liver by Observer 1, accepted as the reference standard. Mask 532 (shown as 532a-532c) (column 3) is the predicted label by the segmentation model. Mask 534 (shown as 534a-534b) (column 5) is the intersection over union (IoU) of the 95% thresholded confidence map (mask 536 (shown as 536a-536c), column 4) and the predicted label (mask 532, column 3). MRE magnitude images are shown in column 1, and measurable liver regions are shown in column 6.

Efficiency Results of Automated (e.g., DL-assisted) and Manual LSM. The 29 MRE magnitude slices were labeled as diagnostic by Observer 1 and were manually processed for a total of 20 min and 19 s for LSM (without accounting for the duration of image transfer, software launch, file organization, etc.), while the DL-assisted method completed the task in under 1 s (p<0.05) (shown in Table 7).

TABLE 7
Total duration of Observer 1 (O1) manual LSM
and DL-assisted LSM for 4 patient cases (n =
29 total slices) in the efficiency evaluation.

| Case (Number | Manual LSM | DL-assisted LSM | DL-assisted LSM |
|---|---|---|---|
| of slices) | by O1 | (GPU-enabled) | (CPU-enabled) |
| 1 (n = 10) | 370 sec | 0.16 sec | 2.27 sec |
| 2 (n = 10) | 425 sec | 0.16 sec | 2.28 sec |
| 3 (n = 6) | 263 sec | 0.09 sec | 1.32 sec |
| 4 (n = 3) | 161 sec | 0.05 sec | 0.65 sec |

Note:
Measurements were obtained with a GPU and a CPU-enabled DL-assisted method.

Discussion. The study achieved high performance of fully automated QC assessment and LSM, with the best-performing QC ensemble model (e.g., classification model and segmentation model) achieving an accuracy of 0.971. The mean LSM error between the DL-assisted method and the reference standard was negligible at 1.9±4.6%. Furthermore, the DL-assisted method, using the segmentation model, allowed LSM in less than 1 s.

The MRE workflow may require the MRI technologists and/or the radiologist to verify image quality during acquisition, which may interrupt workflow, be challenging to less experienced users, and require immediate corrective actions. This underscores the need to automate the QC process in liver MRE, as this exam is increasingly being done.

Compared to previous MRE DL QC [15′], the study utilized MRE magnitude slices and their 2D FFT counterparts to use k-space information instead of CMOEs, resulting in higher average test dataset accuracy (0.919 vs 0.851, respectively). This improvement may be attributed to the clearer artifact detection and reduced noise from no longer having a hashed pattern in the image. Additionally, the reference standard in the study was based on the consensus of multiple experienced experts, enhancing reliability.

The study performed quality checks directly on the MRE magnitude images, which were more closely related to the risk of motion artifacts. The rationale is that if the liver cannot be delineated in the magnitude image, the liver boundary was not visible, indicating that the patient was not following the breathing instructions. On the other hand, if liver delineation can be achieved, but the intersection over union (IoU) between the liver mask and the confidence map had no measurable area, then the cause of nondiagnostic MRE could be due to poor wave propagation, even if the patient remained stationary and followed the breath-hold instructions. The poor wave propagation may be caused by issues such as the detached MRE passive driver tube or improper positioning of the passive drum on the liver.

The exemplary system for automatic ROI delineation and LSM using one or more DL models (e.g., 2D U-Net model) achieved a high concordance (Dice score of 0.87) and a negligible LSM error, making the interpretation of liver stiffness a quick and precise process while covering the largest measurable liver area. When making LSMs, each elastogram was evaluated for ROI placement, which can be done manually or using automated techniques [18′]. Manual ROI measurements, performed with a freehand ROI tool, were time-consuming and required experienced radiologists, especially for heterogeneously fibrotic livers, in which measuring the largest possible liver area provides a more comprehensive stiffness assessment [19′]. This automated method may be useful in performing a low number of MRE exams.

Having a high concordance for the liver segmentation model is important, since the process for LSM differs between radiologists and the DL-assisted method. The DL-assisted method first identifies the liver location and then uses the MRE sequence-generated confidence-thresholded mask to exclude areas with low confidence. In contrast, radiologists visually estimate the liver area on CMOE and assume that the ROI they define can cover most of the measurable liver region.

A limitation of the study was that the CNN models (i.e., DL models) used to identify diagnostic quality did not classify the specific root causes of failed MRE acquisitions, which could be useful for practical decision-making, such as outputting a fag to the technologist to fix hardware issues and repeat imaging. Additionally, the QC model did not incorporate wave data, which could provide deeper insights into shear wave propagation patterns. Issues such as poor diagnostic quality and the inability to draw ROIs for liver delineation from the MRE magnitude images acquired by GRE, along with cases of iron deposition, may restrict the generalizability of the study when applying DL-assisted LSM with liver segmentation using the U-Net model.

Another limitation was the softer liver parenchyma in normal livers (LSM<2.5 kPa), which can attenuate waves and simulate a low-quality examination. The exemplary system and method did not address this situation. Moreover, liver “hot spots” along the dome or around large vessels and masses, resulting from shear wave interference [9′], [20′], [21′], may be incorrectly included in the segmentation maps predicted by exemplary system's model(s). These hotspots can affect the precision of LSM, as they reflected artifacts rather than actual liver stiffness. Further research may be needed to address the limitations described herein and enhance the model's robustness and applicability in diverse clinical scenarios.

Future studies may integrate the developed models into the MRE clinical workflow by deploying the online-trained QC model and LSM DL-assisted methodology into clinical MRI scanners such that they take direct inputs from MRE acquisitions in real-time, and their outputs are included in the report. Additionally, future studies may seek to provide per-patient stiffness analysis with an accompanying automated fibrosis staging. Advantageously, the systems and methods described herein are modular and may be incorporated into existing MRE platforms as illustrated in FIG. 6.

### Discussion

Magnetic resonance elastography (MRE) is a complex imaging method that uses motion encoding to measure and display the mechanical characteristics of tissues within the body. It combines magnetic resonance imaging (MRI) with mechanical vibrations to generate detailed maps of tissue stiffness. Through the generation of these maps, known as elastograms, MRE gathers data on tissue stiffness and is commonly used to noninvasively assess the stage of liver fibrosis, which allows the evaluation of the severity of liver disease and informs clinical management [1],[2].

During the MRE acquisition, two datasets of wrapped phase/wave images with opposite polarities are created. By subtracting the unwrapped datasets from each other, the static background information is eliminated, preserving only the propagating wave information [3]. In addition to phase/wave images, magnitude images (anatomical coverage), confidence maps (used to identify areas where the measurements are reliable), elastograms (quantitative map of tissue stiffness), and confidence map overlaid elastograms (CMOEs) are also created [4].

In MRE, patient-related [e.g., iron deposition, body mass index (BMI), ascites] and hardware-related factors (e.g., MRE driver location, tube disconnection) can lead to failure, estimated to be <6% for 2D GRE and approximately 2% for 2D-EPI sequence, however, reported to be as high as 15.3% with 2D GRE at 3 T [5]-[9]. Additionally, 2D Spin-echo EPI MRE has been estimated to have a failure rate of approximately 21% in patients who have hepatic iron overload [10]. A failed MRE may require images to be repeated. The conventional method for reviewing MRE images involves visual examination by a technologist and/or a radiologist [7], [9], [11]. Due to the large number of images produced during an MRE exam, this can be time-consuming and challenging, especially for inexperienced operators. This process could result in unnecessary additional table time, underscoring the need for a streamlined approach.

Automated artifact detection has been described for various MRI applications, such as identifying wrap-around and Gibbs ringing artifacts using explainable class activation map (CAM)-enabled convolutional neural networks (CNNs) [12], and identifying motion, chemical-shift, and radio-frequency artifacts using a CNN ensemble [13]. Yet, to the best of our knowledge, artifact detection and correction strategies [14], [15], and quality control (QC) methods have not been described for 2D liver MRE using confidence map overlaid elastograms. Such a method that can detect a non-diagnostic exam in real time can improve the current MRE workflow by decreasing the quality assessment time from minutes to seconds.

### Conclusion

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification and in the appended claims, the singular forms “a,” “an,” “the” include plural referents unless the context clearly dictates otherwise. The term “comprising” and variations thereof as used herein, is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for recovering minerals from coal ash, it will become evident to those skilled in the art that the implementations are not limited thereto but are applicable for any other type of purification and/or recovery.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Machine Learning. Various analysis systems can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally, one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., an error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an ANN is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, and depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by down sampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

Example computing device. An example computing device upon which the methods described herein may be implemented can include but is not limited to multiprocessor systems, microprocessor-based systems, minicomputers, embedded systems, and/or distributed computing environments, including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In an example configuration, the computing device includes at least one processing unit and system memory. Depending on the exact configuration and type of computing device, system memory may be volatile (such as random access memory (RAM)), non-volatile (such as read-only memory (ROM), flash memory, etc.), or some combination of the two. The processing unit may be a programmable processor that performs arithmetic and logic operations necessary for the operation of the computing device. The computing device may also include a bus or other communication mechanism for communicating information among various components of the computing device.

Computing devices may have additional features/functionality. For example, computing devices may include additional storage, such as removable storage and non-removable storage, including, but not limited to, magnetic or optical disks or tapes. Computing devices may also contain network connection(s) that allow the device to communicate with other devices. Computing device may also have input device(s), such as a keyboard, mouse, touch screen, etc. Output device(s), such as a display, speakers, printer, etc., may also be included. The additional devices may be connected to the bus in order to facilitate the communication of data among the components of the computing device.

The processing unit may be configured to execute program code encoded in tangible, computer-readable media. Tangible, computer-readable media refers to any media that is capable of providing data that causes the computing device (i.e., a machine) to operate in a particular fashion. Various computer-readable media may be utilized to provide instructions to the processing unit 406 for execution. Examples of tangible, computer-readable media may include, but are not limited to, volatile media, non-volatile media, removable media, and non-removable media implemented in any method or technology for storage of information such as computer-readable instructions, data structures, program modules, or other data. System memory, removable storage, and non-removable storage are all examples of tangible computer storage media. Examples of tangible, computer-readable recording media include but are not limited to an integrated circuit (e.g., field-programmable gate array or application-specific IC), a hard disk, a solid-state device, RAM, ROM, electrically erasable program read-only memory (EEPROM), flash memory or other memory technology, optical storage, or magnetic storage devices.

In an example implementation, the processing unit may execute program code stored in the system memory. For example, the bus may carry data to the system memory, from which the processing unit receives and executes instructions. The data received by the system memory may optionally be stored on the removable storage or the non-removable storage before or after execution by the processing unit.

### Conclusion

Various sizes and dimensions provided herein are merely examples. Other dimensions may be employed.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

While the methods and systems have been described in connection with certain embodiments and specific examples, it is not intended that the scope be limited to the particular embodiments set forth, as the embodiments herein are intended in all respects to be illustrative rather than restrictive.

The following patents, applications, and publications, as listed below and throughout this document, are hereby incorporated by reference in their entirety herein.

### REFERENCE LIST #1

[1] Guglielmo F F, Barr R G, Yokoo T, et al. Liver Fibrosis, Fat, and Iron Evaluation with MRI and Fibrosis and Fat Evaluation with U S: A Practical Guide for Radiologists. Radiographics 2023; 43(6):e220181.
[2] Kennedy P, Wagner M, Castéra L, et al. Quantitative Elastography Methods in Liver Disease: Current Evidence and Future Directions. Radiology 2018; 286(3):738-763.
[3] Hirsch S, Braun J, Sack I. Magnetic Resonance Elastography Physical Background and Medical Applications Preface. Magnetic Resonance Elastography: Physical Background and Medical Applications 2017.
[4] Committee Q M B. MR Elastography of the Liver, Quantitative Imaging Biomarkers Alliance. Profile Stage: Technically Confirmed. Available from: http://qibawiki.rsna.org/index.php/Profiles; Feb. 14, 2022.
[5] Choi S L, Lee E S, Ko A, et al. Technical success rates and reliability of spin-echo echo-planar imaging (SE-EPI) MR elastography in patients with chronic liver disease or liver cirrhosis. European Radiology 2020; 30(3):1730-1737.
[6] Kim D W, Kim S Y, Yoon H M, Kim K W, Byun J H. Comparison of technical failure of MR elastography for measuring liver stiffness between gradient-recalled echo and spin-echo echo-planar imaging: A systematic review and meta-analysis. J Magn Reson Imaging 2020; 51(4):1086-1102.
[7] Guglielmo F F, Venkatesh S K, Mitchell D G. Liver MR Elastography Technique and Image Interpretation: Pearls and Pitfalls. RadioGraphics 2019; 39(7):1983-2002.
[8] Murphy I G, Graves M J, Reid S, et al. Comparison of breath-hold, respiratory navigated and free-breathing MR elastography of the liver. Magnetic Resonance Imaging 2017; 37:46-50.
[9] Wagner M, Corcuera-Solano I, Lo G, et al. Technical Failure of MR Elastography Examinations of the Liver: Experience from a Large Single-Center Study. Radiology 2017; 284(2):401-412.
[10] Liu J, Huang M, Zhang Y, et al. Technical Success and Reliability of Magnetic Resonance Elastography in Patients with Hepatic Iron Overload. Academic Radiology 2024; 31(4):1326-1335.
[11] Pepin K M, Welle C L, Guglielmo F F, Dillman J R, Venkatesh S K. Magnetic resonance elastography of the liver: everything you need to know to get started. Abdominal radiology 2022:1-21.
[12] Jimeno M M, Ravi K S, Jin Z Z, Oyekunle D, Ogbole G, Geethanath S. ArtifactID: Identifying artifacts in low-field MRI of the brain using deep learning. Magnetic Resonance Imaging 2022; 89:42-48.
[13] Lim A, Lo J, Wagner M W, Ertl-Wagner B, Sussman D. Automatic Artifact Detection Algorithm in Fetal MRI. Frontiers in Artificial Intelligence 2022; 5:1-10.
[14] Smith T B. MRI artifacts and correction strategies. Imaging in Medicine 2010; 2(4):445.
[15] Noda C, Ambale Venkatesh B, Wagner J D, Kato Y, Ortman J M, Lima J A. Primer on commonly occurring MRI artifacts and how to overcome them. Radiographics 2022; 42(3): E102-E103.
[16] He K M, Zhang X Y, Ren S Q, Sun J. Deep Residual Learning for Image Recognition. Proc Cvpr Ieee 2016:770-778.
[17] Anand R, Lakshmi S V, Pandey D, Pandey B K. An enhanced ResNet-50 deep learning model for arrhythmia detection using electrocardiogram biomedical indicators. Evol Syst-Ger 2023:83-97.
[18] Pandiar D, Choudhari S, Krishnan R P. Application of Inception V3, SqueezeNet, and VGG16 Convoluted Neural Networks in the Image Classification of Oral Squamous Cell Carcinoma: A Cross-Sectional Study. Cureus J Med Science 2023; 15(11):1-6.
[19] Sunnetci K M, Kaba E, çeliker F B, Alkan A. Comparative parotid gland segmentation by using ResNet-18 and MobileNetV2 based DeepLab v3+architectures from magnetic resonance images. Concurr Comp-Pract E 2023; 35(1):1-14.
[20] Al-Moosawi N M, Khudeyer R S. ResNet-34/DR: A Residual Convolutional Neural Network for the Diagnosis of Diabetic Retinopathy. Inform-Int J Comput 2021; 45(7):115-124.
[21] Sandler M, Howard A, Zhu M L, Zhmoginov A, Chen L C. MobileNetV2: Inverted Residuals and Linear Bottlenecks. 2018 Ieee/Cvf Conference on Computer Vision and Pattern Recognition (Cvpr) 2018:4510-4520.
[22] Mishkin D, Sergievskiy N, Matas J. Systematic evaluation of convolution neural network advances on the imagenet. Computer vision and image understanding 2017; 161:11-19.
[23] McHugh M L. Interrater reliability: the kappa statistic. Biochem Medica 2012; 22(3):276-282.

### REFERENCE LIST #2

[1′] Gines P, Krag A, Abraldes J G, Solà E, Fabrellas N, Kamath P S. Liver cirrhosis. The Lancet 2021; 398(10308):1359-1376.
[2′] Castera L. Noninvasive Methods to Assess Liver Disease in Patients With Hepatitis B or C. Gastroenterology 2012; 142(6):1293-+.
[3′] Smith A D, Porter K K, Abou Elkassem A, Sanyal R, Lockhart M E. Current Imaging Techniques for Noninvasive Staging of Hepatic Fibrosis. Am J Roentgenol 2019; 213(1):77-89.
[4′] Ozkaya E, Kennedy P, Chen J, et al. Precision and Test-Retest Repeatability of Stiffness Measurement with MR Elastography: A Multicenter Phantom Study. Radiology 2024; 311(2):e233136.
[5′] Wagner M, Corcuera-Solano I, Lo G, et al. Technical Failure of MR Elastography Examinations of the Liver: Experience from a Large Single-Center Study. Radiology 2017; 284(2):401-412.
[6′] Kim D W, Kim S Y, Yoon H M, Kim K W, Byun J H. Comparison of technical failure of MR elastography for measuring liver stiffness between gradient-recalled echo and spin-echo echo-planar imaging: A systematic review and meta-analysis. J Magn Reson Imaging 2020; 51(4):1086-1102.
[7′] Gill H E, Lisanti C J, Schwope R B, Kim J, Katz M, Harrison S. Technical success rate of MR elastography in a population without known liver disease. Abdom Radiol 2021; 46(2):590-596.
[8′] Murphy I G, Graves M J, Reid S, et al. Comparison of breath-hold, respiratory navigated, and free-breathing MR elastography of the liver. Magn Reson Imaging 2017; 37:46-50.
[9′] Guglielmo F F, Venkatesh S K, Mitchell D G. Liver MR Elastography Technique and Image Interpretation: Pearls and Pitfalls. Radiographics 2019; 39(7):1983-2002.
[10′] Dzyubak B, Glaser K, Yin M, et al. Automated liver stiffness measurements with magnetic resonance elastography. J Magn Reson Imaging 2013; 38(2):371-379.
[11′] Shire N J, Yin M, Chen J, et al. Test-retest repeatability of MR elastography for noninvasive liver fibrosis assessment in hepatitis C. J Magn Reson Imaging 2011; 34(4):947-955.
[12′] Murphy M C, Manduca A, Trzasko J D, Glaser K J, Huston J, Ehman R L. Artificial neural networks for stiffness estimation in magnetic resonance elastography. Magn Reson Med 2018; 80(1):351-360.
[13′] Scott J M, Arani A, Manduca A, et al. Artificial neural networks for magnetic resonance elastography stiffness estimation in inhomogeneous materials. Med Image Anal 2020; 63.
[14′] Cunha G M, Delgado T, Middleton M S, et al. Automated CNN-Based Analysis Versus Manual Analysis for MR Elastography in Nonalcoholic Fatty Liver Disease: Intermethod Agreement and Fibrosis Stage Discriminative Performance. Am J Roentgenol 2022; 219(2):224-232.
[15′] Nieves-Vazquez H A, Ozkaya E, Meinhold W, et al. Deep Learning-Enabled Automated Quality Control for Liver MR Elastography: Initial Results. J Magn Reson Imaging 2025; 61:985-994.
[16′] Hectors S J, Kennedy P, Huang K H, et al. Fully automated prediction of liver fibrosis using deep learning analysis of gadoxetic acid-enhanced MRI. Eur Radiol 2021; 31(6):3805-3814.
[17′] Ronneberger O, Fischer P, Brox T. (2015) U-net: convolutional networks for biomedical image segmentation. Medical image computing and computer-assisted intervention—MICCAI 2015: 18th International Conference, Munich, Germany, Proceedings, part III 18: Springer. p. 234-241.
[18′] Dzyubak B, Venkatesh S K, Manduca A, Glaser K J, Ehman R L. Automated liver elasticity calculation for MR elastography. J Magn Reson Imaging 2016; 43(5):1055-1063.
[19′] Jhaveri K S, Hosseini-Nik H, Sadoughi N, et al. The development and validation of magnetic resonance elastography for fibrosis staging in primary sclerosing cholangitis. Eur Radiol 2019; 29(2):1039-1047.
[20′] Pepin K M, Welle C L, Guglielmo F F, Dillman J R, Venkatesh S K. Magnetic resonance elastography of the liver: everything you need to know to get started. Abdom Radiol (NY) 2022; 47(1):94-114.
[21′] Srinivasa Babu A, Wells M L, Teytelboym O M, et al. Elastography in Chronic Liver Disease: Modalities, Techniques, Limitations, and Future Directions. Radiographics 2016; 36(7):1987-2006.

What is claimed is:

1. A method comprising:
obtaining magnetic resonance elastography (MRE) imaging data of a subject obtained during a MRE procedure;
determining, via a trained AI classification model, a quality assessment metric of the MRE imaging data, wherein the quality assessment metric corresponds to an indication of a diagnostic quality of the MRE imaging data; and
screening the MRE imaging data based on the quality assessment metric to provide a filtered data set of MRE imaging data classified as diagnostic quality.

2. The method of claim 1, wherein the AI classification model comprises a binary classification model.

3. The method of claim 1, wherein the AI classification model comprises at least one of ResNet18, ResNet34, ResNet50, SqueezeNet, MobileNetV2, or a combination thereof.

4. The method of claim 3, wherein the AI classification model comprises SqueezeNet.

5. The method of claim 1, wherein the AI classification model comprises an explainable AI (XAI) model.

6. The method of claim 5, wherein the XAI model is configured to determine, based on features of a non-diagnostic quality image set, a predicted artifact source.

7. The method of claim 6, further comprising adjusting a parameter for collecting a MRE image based on the predicted artifact source.

8. The method of claim 1, wherein the MRE imaging data comprises MRE magnitude images, 2D Fast Fourier transform (FFT) of MRE magnitude images, or a combination thereof.

9. The method of claim 1, further comprising generating, via a trained AI segmentation model, a segmentation mask corresponding to a region of interest within the filtered data set of MRE imaging data.

10. The method of claim 9, wherein the segmentation mask is subsequently used to determine a measurable stiffness area within the filtered data set of MRE imaging data.

11. The method of claim 10, wherein the measurable stiffness area is determined for the filtered data set of MRE imaging data using an intersection over union (IoU) of the segmentation mask and a thresholded confidence map obtained from the trained AI segmentation model.

12. The method of claim 11, further comprising diagnosing a condition in the subject based on stiffness values within the measurable stiffness area.

13. A system comprising:
a processor; and
a memory having instructions stored thereon, wherein execution of the instructions by the processor causes the processor to:
obtain magnetic resonance elastography (MRE) imaging data of a subject collected during a MRE procedure;
determine, via a trained AI classification model, a quality assessment metric of the MRE imaging data, wherein the quality assessment metric corresponds to an indication of a diagnostic quality of the MRE imaging data; and
screen the MRE imaging data based on the quality assessment metric to provide a filtered data set of MRE imaging data classified as diagnostic quality.

14. The system of claim 13, further comprising:
a MR scanner configured to collect the MRE imaging data of the subject; and
a driver configured to generate shear waves in a tissue of interest of a subject.

15. The system of claim 13, wherein the AI classification model comprises a binary classification model.

16. The system of claim 13, wherein the AI classification model comprises an explainable AI (XAI) model configured to determine, based on features of a non-diagnostic quality image, a predicted artifact source.

17. The system of claim 13, wherein the MRE imaging data comprises MRE magnitude images, 2D Fast Fourier transform (FFT) of MRE magnitude images, or a combination thereof.

18. The system of claim 13, further comprising a trained AI segmentation model configured to generate a segmentation mask corresponding to a region of interest within the filtered data set of MRE imaging data.

19. The system of claim 18, wherein the trained AI segmentation model is further configured to determine a measurable stiffness area within the filtered data set of MRE imaging data.

20. The system of claim 19, wherein the system is further configured to determine the measurable stiffness area for the filtered data set of MRE imaging data using an intersection over union (IoU) of the segmentation mask and a thresholded confidence map obtained from the trained AI segmentation model.
